(19) United States
(12) Patent Application Publication
(10) Pub. No.: US 2017/0354744 A1
(43) Pub. Date: Dec. 14, 2017

(54) METHOD OF SYNTHESIZING OF ANTIBODY CONJUGATES USING AFFINITY RESINS

(71) Applicant: ADC Biotechnology Ltd., St. Asaph (GB)

(72) Inventors: David John Evans, Chester (GB); Colin Martin McKee, Colwyn Heights (GB)

(21) Appl. No.: 15/523,117

(22) PCT Filed: Oct. 27, 2015
(86) PCT No.: PCT/GB2015/053222

§ 371 Date: Apr. 28, 2017

(30) Foreign Application Priority Data
Oct. 28, 2014 (GB) 1419184.5

(51) Int. Cl. A61K 47/69; C07K 1/04; A61K 47/68; C07K 16/28; C07K 1/13; C07K 16/40

(52) U.S. Cl. CPC A61K 47/6913; C07K 16/2863; C07K 1/13; C07K 1/042; A61K 47/6855; A61K 47/6803; A61K 47/6849; C07K 16/40

(57) ABSTRACT

Disclosed is a solid phase method of synthesising biomolecule-effector-conjugates and biomolecule-reporter-conjugates. In particular, this invention relates to a solid phase method of synthesising antibody-effector-conjugates and antibody-reporter conjugates. This invention also relates to intermediate methods of producing immobilised, chemically modified biomolecules, e.g., antibodies.

This invention relates to a solid phase method of synthesising biomolecule-effector-conjugates and biomolecule-reporter-conjugates. In particular, this invention relates to a solid phase method of synthesising antibody-effector-conjugates and antibody-reporter conjugates. This invention also relates to intermediate methods of producing immobilised, chemically modified biomolecules, e.g. antibodies.

In addition to the above methods, the invention relates to various uses of capture resins.

The invention also relates to intermediate products, compositions of the methods of the invention and biomolecule-effector-conjugates and biomolecule-reporter-conjugates, in addition to other subject matter.

### BACKGROUND

Antibodies are a class of highly specialised biomolecules that bind specifically to cells expressing their cognate antigen. These antigens are most commonly cell surface proteins, polypeptides or carbohydrates but antigens may also be lipids, nucleic acids or even small molecules such as neurotransmitters. A particular antibody molecule can only interact with a small region of an antigen and in the case of a polypeptide this is generally a sequence consisting of about 5-12 amino acids. This antigen region can be continuous or it can be distributed in different regions of a primary structure that are brought together because of the secondary or tertiary structure of the antigen. The region of an antigen that is recognized by an antibody is called an epitope. Antibodies bind antigens through weak chemical interactions—bonding is essentially non-covalent. The interaction of antibody with the antigen is known to occur via electrostatic interactions, hydrogen bonds, van der Waals forces and hydrophobic interactions. The specificity of antibodies or immunoglobulins has been exploited for the construction and application of antibody conjugates (immuno conjugates) for therapeutic, diagnostic and most recently theranostic purposes.

Antibodies represent the ideal transportation ‘vehicles’ for applications that require the regiospecific delivery of a payload. An antibody with a discreet payload is defined as a conjugate, more specifically an antibody conjugate. The inherent specificity of an antibody for its cognate antigen has been most successfully exploited within Oncology therapy wherein antibody conjugates have prevalence as an alternative, more efficacious therapy in contrast to traditional chemotherapy.

Conventional chemotherapy employs potent small drug molecules, designed to eliminate fast-proliferating tumour cells. However, this conventional therapy does not discriminate between fast-proliferating tumour cells and fast proliferating healthy cells. Therefore, traditional chemotherapy is burdened with many undesirable side effects to the patient. Furthermore, the therapeutic window for conventional chemotherapy drugs is very limited. Often a large dosing of the drug is required to achieve the desired efficacious effect but this is paralleled with a rapid ascension towards the maximum tolerated dose which if exceeded causes toxicity for the patient. In the preceding decade a more precise, targeted therapy approach using antibody conjugates has been investigated in a concerted attempt to improve efficacy whilst simultaneously increasing the therapeutic window by reducing systemic toxicity.

Investigators have explored 4 main avenues using antibodies to target cytotoxic payloads to malignant cells. These include: antibody-protein toxins (or antibody fragment-protein toxin fusion) conjugates, antibody-chelated radionuclide conjugates, antibody-small-molecule drug conjugates, and antibody-enzyme conjugates administered along with small-molecule prodrugs that require metabolism by the conjugated enzyme to release the activated species. Only antibody radionuclide conjugates and Antibody Drug Conjugates (ADCs) have reached the regulatory approval stage (B. Teicher et al, Clinical Cancer Research; Oct. 15, 2011, 17; 6389).

Antibody Drug Conjugates (ADCs) are a broad class of antibody conjugates that combine the discrete targeting ability of antibodies to precisely deliver a payload capable of having a biological effect. Typically for ADCs the antibody is ‘armed’ with an ultra-potent cytotoxic drug payload facilitating a targeted delivery of the payload specifically to the site of disease. Various linker strategies and a variety of cytotoxic drug payloads; with different modalities, can be conjugated to antibodies. Once at the site of disease the drug payload can deliver the desired cytotoxic cell killing action sparing localised healthy cells. In contrast to traditional chemotherapy drugs, ADCs are designed to increase the efficacy of therapy and reduce systemic toxicity by greatly enhancing the therapeutic window. ADC constructs may be therapeutic against many diseases but recent clinical trials with commercial ADCs Adcetris (Brentuximab Vedotin, Seattle Genetics/Millennium) & Kadcyla (Trastuzumab Emtansine, Genentech/Roche) have demonstrated a particular prevalence within oncology.

In principle, a suitable cytotoxic drug payload for an ADC can be any moiety defined as a L01 ATC molecule (‘Anatomical Therapeutic Chemical Classification System’ where L01 is a subgroup defining antineoplastic and immunomodulating agents, defined by WHO Collaborating Centre for Drug Statistics Methodology). Alternatively, other moieties that may be categorised as suitable payloads for ADCs may be simply defined as anything that is toxic to cells once internalised. Most moieties falling in the latter category would lack sufficient potency to be effective. Hence, there is an industry trend to identify and exploit ‘ultra-potency’ materials. At the time of writing there are currently >33 ADCs in clinical trials and a further >250 ADCs in early phase evaluation.

Expert reviews on the rationale, design and effectiveness of immunotoxin and ADC research can be found within: J. Adair et al, Expert Opin. Biol. Ther., 2012, 12(9): P 1191-206, G. Casi et al, Journal of Controlled Release, 2012, 161, 2, P 422-428, F. Dosio et al, Toxins, 2011, 3, P 848-883 and S. Panowski et al mAbs 2014, 6:1, 34-45.

Complimentary to the use of ADCs as a mode of therapy, antibody conjugates have been broadly utilised for a variety of different purposes. Antibody conjugates in which antibodies are covalently attached to reporter groups have been used in diagnostic assays and analysis techniques. These reporter payloads can be labels, tags and probes. These reporter groups can contain functional groups that provide sensitive detectability through intrinsic chemical or atomic properties such as imaging agents, chelation agents, fluorescence, radioactivity, etc. Antibody conjugates of this type are designed to exhibit an affinity and specificity for a particular target antigen, such that the antibody conjugate is selectively delivered to the antigen presenting moiety and is maintained there for a length of time. Using payloads such as imaging agents the antibody conjugate can be used to illuminate a specific antigen presenting moiety either in-vitro or in-vivo.

Monoclonal antibodies can be raised to selectively detect the presence of a given substance. Once antibodies are produced and appropriately conjugated with a reporter payload they can be used to detect the presence of this substance. Within biochemistry, antibody conjugates that are derivatised with reporter payloads have found extensive utility in the field of diagnostics such as immunohistochemistry, where the antibody conjugates are used to detect specific antigens in fixed tissue sections and immunofluorescence testing; in which antibody conjugates detect antigens in frozen tissue sections or live cells.

Antibodies can be raised to haptens of a non-biological nature. Antibody conjugates derivatised with fluorescent tags have been used successfully in such diverse challenges as trace detection of explosives (S, Ramin et al, J. Mol. Recognit., 2012; 25: 89-97), pesticide residues and chemical pollutants.

Most commonly, antibody conjugates with reporter molecules are employed for immunoassays such as enzyme-linked immunosorbent assays (ELISA) or radioimmuno assays (RIA). There are many variants of these approaches but all are based on a similar idea. A bound antigen on a solid support or surface can be detected by reacting it with its cognate antibody. The antibody-complex can then be quantitated by reacting the antibody with either a secondary antibody or by incorporating a label directly onto the primary antibody making an antibody conjugate. Alternatively, an antibody can be bound to a solid surface and the antigen added. A second antibody that recognizes a distinct epitope on the antigen can then be added and detected. This is frequently called a ‘sandwich assay’ and can frequently be used to avoid problems of high background or non-specific reactions.

A number of solution-phase methods can be used to manufacture biomolecule-drug-conjugates, e.g. antibody-drug-conjugates (ADCs). However, solution phase methods are themselves wasteful in terms of generating large volumes of waste and are problematic in terms of aggregation of the biomolecule-drug-conjugates during synthesis.

The first step in a solution-phase method for manufacturing biomolecule-drug-conjugates generally involves chemical modification or activation of the biomolecule. For example, where the biomolecule is an antibody, the antibody can be ‘chemically modified’ or ‘activated’ by reducing or partially reducing the antibody. A suitable process for partial reduction of antibodies is given in “Bioconjugate Techniques”, page 96/97, Greg T. Hermanson, Academic Press; 2nd edition, 2008, ISBN-13: 978-0123705013. A reducing agent such as TCEP is generally employed in the reduction process.

After chemical modification or activation of the antibody, e.g. reduction, the next step is to remove any excess activation/chemical modification agent, e.g. excess reducing agent. This step is very time consuming as it is often necessary to run the sample through a separation column multiple times. This can also be problematic in terms of degradation if stability of the biomolecule is an issue. Alternatively a diafiltration step can be applied but this can lead to loss of material during processing.

After the above purification step, the chemically modified/activated, e.g. reduced, antibody is then conjugated with a drug moiety. The major problem with this step is the high probability of aggregation of the biomolecule-drug-conjugate. This is particularly problematic when highly hydrophobic drug payloads are employed in the process.

Aggregation is a major problem as it can lead to unusable biomolecule-drug-conjugates. In the best case scenario, biomolecule-drug-conjugates contaminated with biomolecule-drug-conjugate aggregates must be further purified to remove the aggregates, which is both time consuming and very wasteful. A large proportion of the drug will be lost during purification as it forms part of the aggregated biomolecule-drug-conjugate. In the worst case the entire batch of biomolecule-drug-conjugate contaminated with biomolecule-drug-conjugate aggregate to such a high degree it is entirely unusable and must be disposed of. The step of purification of the biomolecule-drug-conjugate can be very time consuming as it is often necessary to run the conjugate through the column multiple times.

Oncologists have been working on harnessing target-specific monoclonal antibodies to deliver cytotoxic drugs to the site of tumours as long as monoclonal antibodies have existed; nearly three decades. Up until now three classes of toxin have dominated the field. Namely, calicheamicins, maytansines and auristatins. These cytotoxic drug classes are all typically hydrophobic in nature. When conjugated to an antibody their presence increases the overall hydrophobicity of the antibody significantly and in some cases to the extent that hydrophobic interactions between conjugates leads to conjugate aggregation (Y. Adem et al, Bioconjugate Chem. 2014, 25, 656-664). The order of significance of this issue is Calicheamicin>Maytansine>Auristatin based on the knowledge that the processes for both Mylotarg and CMC-544 contain chromatographic aggregate removal steps.

Approximately 50% of maytansine processes contain aggregate removal steps and very few auristatin processes contain aggregate removal steps.

More recently, cytotoxic toxin payloads based on duocarmycin's (www.syntarga.com), pyrollebenzodiazepene (PBD) dimers (www.spirogen.com) and alpha-amanitin's (www.heidelberg-pharma.com) have been conjugated to antibodies and are undergoing pre-clinical evaluation. These new classes of toxin are even more hydrophobic than their predecessor cytotoxin drug classes and are more prone to aggregation when conjugated to antibodies via stochastic means (S. Jeffrey et al, Bioconjugate Chem. 2013, 24, 1256-1263).

Aggregation is a cause of physical instability and can be a limiting stability parameter for an antibody conjugate product such as an ADC. Aggregate content should be kept to a bare minimum in a product because these materials have important efficacy and toxicity effects on patients (M. Manning et al, Pharm. Res; 2010, 27, 544-75).

Significant efforts have been focussed on modulation of the hydrophobicity of the drug by incorporating hydrophilic linkers (Zhao et al, J. Med. Chem., 2011, 54, 10, 3606-3623). Where aggregate formation cannot be controlled developers have relied on well-known techniques for aggregate removal from protein based therapeutics. These include a range of different chromatographic separations including ion exchange, hydrophobic interaction, hydroxyapatite (WO03/057163) and others well known to those in the art (A. Wakankar et al MAbs, 2011 March-April; 3, 2, 161). Most recently tangential flow filtration purification has found favour (WO2006086733). Undertaking such purification techniques has the result of achieving adequate product quality but is challenging and is often at the expense of process yield. When working with antibodies and antibody based therapeutics in the context of manufacturing, physical loss of material through ambiguous, incidental side reactions or unwanted physiochemical interactions through aggregate formation has a hugely significant financial impact.

Manning et al (Pharm. Res., 2010, 27, 4, 544) defines aggregates as (i) rapidly reversible non-covalent small oligomers (dimer, trimer, tetramer, etc.); (ii) irreversible non-covalent oligomers; (iii) covalent oligomers (e.g., disulfide-linked); (iv) large aggregates (>10mer's), which could be reversible if non-covalent; (v) very large aggregates (50 nm to 3000 nm diameter), which could be reversible if non-covalent; and (vi) visible particulates (‘snow’), which are probably irreversible. Aggregation can arise from non-covalent interactions or from covalently linked species. When comparing the biological activity of monomeric antibody conjugates with that of corresponding aggregates the presence of these high molecular weight species can significantly impair the potency of the conjugate. In such cases product efficacy may be compromised (M. Vazquez-Rey et al, Biotechnology and Bioengineering, 2011, Vol. 108, 7, 1495).

Aggregate formation has a direct and negative effect on the monomer purity in a biomolecule or antibody conjugate. Aggregation is a major problem as it can lead to unusable biomolecule and antibody conjugates. If aggregation effects are prevalent, a large proportion of the effector or reporter payload will be lost during purification as it is a component part of the conjugate. In the worst case, the entire batch of conjugate will be contaminated with aggregate to such a high degree it is entirely unusable and unsuitable for multi-pass purification and thus must be disposed of.

It is understood that the degree of aggregation in an antibody drug conjugate is directly proportional to the extent of hydrophobic drug toxin incorporated onto the antibody. For stochastic conjugations in which an antibody is derivatised with a cytotoxic payload the resultant conjugate will comprise of a spread of Drug Antibody Ratio (DAR) species. When targeting a DAR of 4 the spread of DAR species will be Gaussian and typically between 0 and 8. For conjugates prone to aggregate these higher DAR species will typically be present in the aggregate. In manufacturing this phenomenon has the effect of reducing the DAR of the overall conjugate causing the process to fall away from the target DAR specification. Thus, aggregation has a negative impact on achieving target DAR for the antibody conjugate.

Participants of the ADC World conference were surveyed to find out what they viewed as the greatest CMC hurdles for ADC development. Over half of participants (52%) regarded unwanted aggregation as the major issue. 40% Of participants also highlighted concern over control of DAR with 20% alerted to the removal of superfluous unbound drug from the conjugate product material (Antibody-Drug Conjugates Industry Outlook 2014 “Insight and analysis of the major trends, challenges and opportunities within the ADC field” Hanson Wade, page 10).

Furthermore, it is difficult to attach a reporter or effector group to an antibody via a stochastic conjugation process without interfering with the antigen binding capacity and/or specificity and thus reducing the effectiveness of the antibody conjugate.

There is an increasing FDA demand for ADC homogeneity in which developers are encouraged to evidence the precise location of conjugation event. To address these demands current state-of-the-art antibody conjugation techniques implement site-specific conjugation techniques in which the conjugation event occurs only at engineered cysteine residues (ThioMab, Genentech), unnatural amino acids (Azido, SynAfix) or via enzymatic means (SMARTag, Redwood Bioscience) via engineered markers at specific locations on the antibody. Site-specific conjugation has resulted in greater homogeneous ADC production through the control of the drug antibody ratio (DAR). Improving the DAR has positive effects on the conjugate stability, in vivo tolerability, pharmacokinetic (PK) properties & efficacy.

Typically a site-specific conjugation technique targets a low DAR, typically DAR 2. To realise efficacy and achieve the therapeutic window the cytotoxic payload must be of exquisite potency as the number of conjugation events per antibody is limited. Typically cytotoxic payloads of such exquisite potency are highly hydrophobic in nature and thus are prone to aggregation effects. Despite these advantages of site-specific conjugation the issue of aggregation still prevails.

For all antibody conjugation techniques with a propensity to aggregate, conjugation processes are undertaken at relatively low concentrations, typically around 1 g/L. The use of dilute solution phase methods is a direct approach to attempt to circumvent aggregation effects. However, even at such concentrations aggregation effects can still be observed and detrimental to the product quality which necessitates purification of the entire process batch. The use of dilute solutions for manufacturing and additional process media for purification amasses large quantities of waste solvent which must be disposed of as cytotoxic waste. Manufacturing processes that avoid such accumulation of waste are therefore more environmentally favourable and efficient.

Accordingly, the conventional solution-phase processes for manufacturing biomolecule-effector-conjugates or biomolecule-reporter-conjugates are beset with difficulties and it would be desirable to provide an improved process for manufacturing biomolecule-effector-conjugates or biomolecule-reporter-conjugates.

The present invention addresses one or more of the above issues inherent within the conventional solution-phase methods.

### SUMMARY OF THE INVENTION

In our previous patent, WO2012140433, we describe the concept and methodology of reversibly binding a biomolecule to a substrate using a support bearing certain 1,3-diketone, 1,3-ketothioesters or 1,3-ketoamides to form a covalently bound biomolecule. These covalently bound biomolecules may be purified and/or modified whilst bound to the support and then released in a purified/modified form. Whilst covalently bound the biomolecule or modified biomolecule may be modified with a drug to form a covalently bound biomolecule-drug-conjugate. The bound biomolecule or biomolecule-drug-conjugate can be released from the support on demand under mild chemical conditions in which the integrity of the biomolecule is preserved. The technology described therein enables the reliable and repeatable purification and processing of biomolecules and biomolecule-drug-conjugates in a manner not previously possible by conventional methods.

The particular technology employed in WO2012140433 utilises a diketone-type group which allows for the facile binding of the biomolecule to a suitable derivatised solid phase support. The covalent binding of the biomolecule is selective and proceeds through primary amine groups present on lysine residues or the N-alpha terminal portion of the amino acid sequence. The resulting vinylogous amide (enamine) is stabilised through the formation of a 6-membered ring through hydrogen bonding. The technology is selective for primary amines as the stabilisation of the enamine adduct with primary amines facilitates the formation of the 6-membered ring which is stabilised through said hydrogen bond.

The technology described in WO2012140433 can be differentiated from the technology described herein by several means. Firstly by the concept by which the biomolecule is bound/immobilised. WO2012140433 describes a covalent bond means of binding the biomolecule to a suitable derivatised solid phase support. Herein, the technique of biomolecule immobilisation is based upon affinity interactions between the biomolecule and the suitably derivatised solid support.

Secondly, the diketone based solid supports of WO2012140433 specifically bind primary amine groups. Therefore, in a conjugation reaction using the technique from WO2012140433 and in which any amine containing reporter group or effector group is introduced to a bound biomolecule-solid support complex, the amine containing reporter or effector group may be competitively bound to the diketone support through covalent enamine bond formation. Consequently, for these particular reagents, there would be a negative impact on process efficiency using the process of WO2012140433 due to undesirable sequestering of the reporter or effector moiety (in addition to the desired biomolecule) during the conjugation step. The limitation of the process described in WO2012140433 is only apparent when the process incorporates a primary amine containing effector/reporter compound. In WO2012140433 there is no competitive bonding effect for the reporter or effector groups from the derivatised solid support as the technology relates to affinity bonding of the biomolecule to the solid support.

Thirdly, the diketone based supports of WO2012140433 should avoid buffer compositions with amino based excipients such as amino acids, tris(hydroxymethyl)aminomethane (Tris), proteins, animal derived hydrolysed gelatins, etc. (see T. Kamerzell et al, Advanced Drug Delivery Reviews, 2011, 63, 1118 for a review of typical excipients). This is because the primary amine groups present in the amino acid based excipients will behave as competitive inhibitors with the biomolecule for bonding to the derivatised solid support. The issue is most prevalent when initially immobilising the biomolecule to the diketone solid support therefore the use of such excipients should be avoided. The removal of amine functional excipients can be achieved by processing the biomolecule batch through buffer exchange. As will be apparent from the discussion below, the process of the present invention has no such limitations with amine based excipients as the derivatised solid support has no such affinity for primary amine based excipients.

The methods described in WO2012140433 for the solid phase assembly and release of biomolecule-drug-conjugates employ the bonding of the biomolecule to the diketone functionalised solid support. In WO2012140433 the bonding of the biomolecule to the diketone functionalised support is stochastic. In practice for an antibody, bonding may occur to the diketone functionalised solid support through one or a plurality of solvated surface accessible lysyl residues. On biomolecules such as antibodies, lysine residues are abundant, widely distributed and easily modified because of their nucleophilic reactivity plus their location on the surface of the antibody. WO2012140433 describes a random process wherein accessible, solvated lysine residues from the biomolecule are bonded to the solid support. In any particular antibody clone, lysines may occur prominently within the antigen binding site. In a stochastic process there is the potential that lysine residues within the antigen binding site may be involved with the bonding process. As will be apparent from the discussion below, in the process of the present invention immobilisation of the biomolecule to the solid support is not specific with lysine residues therefore the integrity of the antigen binding site is maintained.

Biomolecule-effector-conjugates and biomolecule-reporter-conjugates may contain either effector groups or reporter groups that are proteinaceous in nature, such as proteins, enzymes, peptides, amino acids, etc. These proteinaceous effector or reporter groups may contain solvated, accessible lysine residues. Therefore, using the technique described in WO2012140433 proteinaceous effector or reporter groups may be competitively sequestered by unreacted diketone functionalities on the solid support (by a process akin to that described above for primary amines). This would negatively affect the stoichiometry of the conjugation reaction resulting in conjugates having lower than anticipated ratios of effector or reporter groups incorporated onto the biomolecule. There is also the possibility that the proteinaceous effector group or reporter group may bond to the diketone support whilst existing as a bonded biomolecule-effector-conjugate or biomolecule-reporter-conjugate. This would in effect be 2 levels of bonding to the diketone support. This may require more forcing release conditions in order to cleave the biomolecule-effector-conjugates or biomolecule-reporter-conjugates from the solid support. The forcing conditions may be attributed to an increased number of bonding points between the solid support and the conjugates. As will be apparent from the discussion below, in the process of the present invention limitation is not apparent. There is no competitive bonding effect for proteinaceous reporter or effector groups (excluding antibody-based reporter and effector groups) from the derivatised solid support.

The functionalised 1,3-diketo functionalised supports as described in WO2012140433 exist in equilibrium between keto and enol tautomeric forms. This enolization enables 1,3-diketo functionalised supports to chelate to a range of metals including Tin, Zirconium, Zinc, aluminium, Lead, Copper, etc. (A. Gambero et al, Journal of Colloid and Interface Science, 1997, 185, 313). For biomolecule-effector-conjugates or biomolecule-reporter-conjugates assembled using WO2012140433 in which the effector or reporter group is a complexing agent containing metal there may be a competitive binding effect from the diketo functional support for the metal. This may result in the diketo groups scavenging the metal from the complexing agent resulting in loss of the metal from the assembled conjugate. Alternatively difficulty in releasing the biomolecule-complexing agent containing metal-conjugates from the solid support could be envisaged due to an increase in affinity for the metal contained in the conjugate by the diketo functionalised solid support. As will be apparent from the discussion below, in the process of the present invention this limitation is not apparent. There is no competitive metal scavenging from the solid supports described within the context of this invention.

The 1,3-diketo functionalised supports as described in WO2012140433 facilitate the synthesis of biomolecule-effector-conjugates or biomolecule-reporter-conjugates using a solid phase technique. The supports are designed for single use as upon cleavage of the assembled biomolecule-conjugates the diketo groups are derivatised to a new species by scavenging the chemical key that effected release of the biomolecule-conjugates. In the context of WO2012140433 the diketo functionalised supports cannot be recycled. As will be apparent from the discussion below, the process of the present invention allows for recyclability of the support; the support can be reused multiple times without significant deterioration in process efficiency.

Accordingly, the process described in WO2012140433 has some limitations when synthesising a biomolecule-effector conjugate or biomolecule-reporter-conjugate via certain embodiments. It would be desirable to remove such limitations to provide a more robust process for manufacturing biomolecule-effector conjugates or biomolecule-reporter-conjugates.

The present invention addresses one or more of the above issues not anticipated but inherent within the scope of WO2012140433.

One object of the present invention is to provide a conjugate of a diagnostic principle to a biomolecule wherein a plurality of molecules of the diagnostic principle are attached to the biomolecule, for enhanced diagnostic effect at the target site.

One object of the present invention is to provide a conjugate of a diagnostic principle to a targeting antibody, modified antibody or antibody fragment wherein a plurality of molecules of the diagnostic principle are attached to the antibody, modified antibody or antibody fragment for enhanced diagnostic effect at the target site.

Another object of the invention is to provide a conjugate of a plurality of molecules of a diagnostic principle to an antibody, modified antibody or antibody fragment wherein the conjugate has substantially the same immunoreactivity as intact antibody, modified antibody or antibody fragment.

Another object of the present invention is to provide a conjugate of a plurality of detectable labels of a reporter principle to an antibody, modified antibody or antibody fragment wherein the conjugate is for use as a reagent for immunoassays or immunohistology.

Another object of the invention is to provide a biomolecule-reporter-conjugate comprising a plurality of chelator reporter groups for use as a targeted imaging agent for scintigraphic or magnetic resonance imaging.

One object of the present invention is to provide a conjugate of an effector principle to a biomolecule wherein a plurality of molecules of the effector principle are attached to the biomolecule for enhanced effector effect at the target site

One object of the present invention is to provide a conjugate of an effector principle to a targeting antibody, modified antibody or antibody fragment wherein a plurality of molecules of the effector principle are attached to the antibody, modified antibody or antibody fragment for enhanced effector effect at the target site.

Another object of the invention is to provide a conjugate of a plurality of molecules of an effector principle to an antibody, modified antibody or antibody fragment wherein the conjugate has substantially the same immunoreactivity as intact antibody, modified antibody or antibody fragment.

Another object of the present invention is to provide a conjugate of an antibiotic effector payload to an antibody, modified antibody, antibody fragment wherein the conjugate does not appreciably bind to non-target cells or tissues, and wherein the targeted conjugate can enter site of infection or release its effector principle at the target site and achieve its antibiotic effect at the target, while minimizing the systemic side effects of the antibiotic effector payload.

Another object of the present invention is to provide a conjugate of an anti-inflammatory effector payload to an antibody, modified antibody, antibody fragment wherein the conjugate does not appreciably bind to non-target cells or tissues, and wherein the targeted conjugate can enter site of infection or release its effector principle at the target site and achieve its anti-inflammatory effect at the target, while minimizing the systemic side effects of the anti-inflammatory effector payload.

Another object of the present invention is to provide a conjugate of an autoimmune effector payload to an antibody, modified antibody, antibody fragment wherein the conjugate does not appreciably bind to non-target cells or tissues, and wherein the targeted conjugate can enter site of infection or release its effector principle at the target site and achieve its autoimmune effect at the target, while minimizing the systemic side effects of the autoimmune effector payload.

Another object of the present invention is to provide a conjugate of an anti-neurodegenerative effector payload to an antibody, modified antibody, antibody fragment wherein the conjugate does not appreciably bind to non-target cells or tissues, and wherein the targeted conjugate can enter site of infection or release its effector principle at the target site and achieve its anti-neurodegenerative effect at the target, while minimizing the systemic side effects of the anti-neurodegenerative effector payload.

Another object of the invention is to provide an antibody conjugate comprising a plurality of boron addends, suitable for use as a neutron activation therapy agent.

Another object of the invention is to provide an antibody conjugate comprising a plurality of gadolinium addends, suitable for use as a neutron capture therapy agent.

The present invention satisfies one or more of the above aims.

Method of Synthesising a Biomolecule-Effector-Conjugate:

In accordance with the present invention there is provided a method of synthesising a biomolecule-effector-conjugate, the method comprising:

a) optionally contacting a biomolecule with a chemical modification agent, enzymatic modification agent or activating agent to provide a chemically modified, enzymatically modified or activated biomolecule;
b)

(i) when step (a) is carried out, contacting the chemically modified, enzymatically modified or activated biomolecule of step (a) with a capture resin comprising a non-peptide based Protein A, Protein G or Protein L mimetic biomolecule capture moiety under conditions suitable to immobilise the chemically modified, enzymatically modified or activated biomolecule and therefore provide an immobilised chemically modified, enzymatically modified or activated biomolecule; or

(ii) when step (a) is not carried out, contacting a biomolecule with a capture resin comprising a non-peptide based Protein A, Protein G or Protein L mimetic biomolecule capture moiety under conditions suitable to immobilise the biomolecule and therefore provide an immobilised biomolecule;

c) optionally contacting the immobilised chemically modified, enzymatically modified or activated biomolecule of step (b)(i) or the immobilised biomolecule of step (b)(ii) with a chemical modification agent, enzymatic modification agent or activating agent to provide an immobilised chemically modified, enzymatically modified and/or activated biomolecule;
d) optionally washing the immobilised chemically modified, enzymatically modified or activated biomolecule of step (b)(i); the immobilised biomolecule of step (b)(ii); or the immobilised chemically modified, enzymatically modified and/or activated, immobilised biomolecule of step (c) with buffer to remove superfluous or unreacted chemical modification agent, enzymatic modification agent or superfluous or unreacted activating agent,
e) optionally repeating step (c) and step (d);
f) optionally contacting an effector component with a chemical modification agent, enzymatic modification agent or activating agent to provide a chemically modified, enzymatically modified or activated effector component;
g)
(i) when step (f) is carried out, contacting the immobilised biomolecule or the immobilised chemically modified, enzymatically modified and/or activated biomolecule with the chemically modified, enzymatically modified or activated effector component of step (f) to form an immobilised biomolecule-effector-conjugate; or
(ii) when step (f) is not carried out contacting the immobilised biomolecule or the immobilised chemically modified, enzymatically modified and/or activated biomolecule with an effector component to form an immobilised biomolecule-effector-conjugate;
   h) optionally washing the immobilised biomolecule-effector-conjugate of step (g) with buffer to remove superfluous or unreacted reagents, to provide a purified immobilised biomolecule-effector conjugate;
   i) releasing the purified biomolecule-effector-conjugate from the capture resin; wherein the biomolecule is an antibody, modified antibody or antibody fragment.

Method of Synthesising a Biomolecule-Reporter-Conjugate:

In accordance with the present invention there is provided a method of synthesising a biomolecule-reporter-conjugate, the method comprising:

a) optionally contacting a biomolecule with a chemical modification agent, enzymatic modification agent or activating agent to provide a chemically modified, enzymatically modified or activated biomolecule;
b)
(i) when step (a) is carried out, contacting the chemically modified, enzymatically modified or activated biomolecule of step (a) with a capture resin comprising a non-peptide based Protein A, Protein G or Protein L mimetic biomolecule capture moiety under conditions suitable to immobilise the chemically modified, enzymatically modified or activated biomolecule and therefore provide an immobilised chemically modified, enzymatically modified or activated biomolecule; or
(ii) when step (a) is not carried out, contacting a biomolecule with a capture resin comprising a non-peptide based Protein A, Protein G or Protein L mimetic biomolecule capture moiety under conditions suitable to immobilise the biomolecule and therefore provide an immobilised biomolecule;
   c) optionally contacting the immobilised chemically modified, enzymatically modified or activated biomolecule of step (b)(i) or the immobilised biomolecule of step (b)(ii) with a chemical modification agent, enzymatic modification agent or activating agent to provide an immobilised chemically modified, enzymatically modified and/or activated biomolecule;
   d) optionally washing the immobilised chemically modified, enzymatically modified or activated biomolecule of step (b)(i); the immobilised biomolecule of step (b)(ii); or the immobilised chemically modified, enzymatically modified and/or activated, immobilised biomolecule of step (c) with buffer to remove superfluous or unreacted chemical modification agent, enzymatic modification agent or superfluous or unreacted activating agent,
   e) optionally repeating step (c) and step (d);
   f) optionally contacting an effector component with a chemical modification agent, enzymatic modification agent or activating agent to provide a chemically modified, enzymatically modified or activated effector component;
   g)
(i) when step (f) is carried out, contacting the immobilised biomolecule or the immobilised chemically modified, enzymatically modified and/or activated biomolecule with the chemically modified, enzymatically modified or activated reporter component of step (f) to form an immobilised biomolecule-reporter-conjugate; or
(ii) when step (f) is not carried out contacting the immobilised biomolecule or the immobilised chemically modified, enzymatically modified and/or activated biomolecule with a reporter component to form an immobilised biomolecule-reporter-conjugate;
   h) optionally washing the immobilised biomolecule-reporter-conjugate of step (g) with buffer to remove superfluous or unreacted reagents, to provide a purified immobilised biomolecule-reporter conjugate;
   i) releasing the purified biomolecule-reporter-conjugate from the capture resin;
   wherein the biomolecule is an antibody, modified antibody or antibody fragment.

A key feature of the above methods of the invention is that the capture resin employed in the process is able to immobilise the biomolecule in a consistent and reproducible manner. Consistent immobilisation of the biomolecule to the capture resin should result in reduced variation in the resulting biomolecule-effector-conjugate or biomolecule-reporter-molecule produced by the above method. For example, the variation in the point at which the effector or reporter component is attached to the immobilised biomolecule might be reduced, thus leading to a more consistent point of attachment between the effector or reporter component and the immobilised biomolecule. Such an improvement in regio-specificity would be desirable in terms of improving the consistency of the resulting biomolecule-effector-conjugate or biomolecule-reporter-conjugate products.

A desirable feature of the above method is that the immobilisation of the biomolecule reduces intermolecular interaction and therefore aggregation. For complex biomolecules with tertiary structure such as antibodies immobilisation to a capture resin minimises unfolding through the multipoint attachment of the biomolecule to the capture resin. Therefore, the number of attachment points between the resin and the biomolecule correlates well with an enhancement of stability achieved through the immobilisation step.

The employment of a non-peptide-based Protein A, Protein G or Protein L mimetic as the biomolecule capture moiety, as opposed to the employment of the parent Protein A, Protein G or Protein L as the biomolecule capture moiety, may lead to a relative improvement in consistency in the immobilisation of the biomolecule due to increased regio-specificity of the mimetic verses the conventional Protein A, Protein G or Protein L based systems. In cases in which the regio-specificity of the immobilisation of biomolecules to proteins is low, the employment of the parent Protein A, Protein G or Protein L as the biomolecule capture moiety would inherently result in variable immobilisation of the biomolecule to the capture resin. For example, the parent Protein A, Protein G or Protein L may exhibit non-specific binding via other sites on the protein which may complicate the overall interaction. As explained above, consistent immobilisation of the biomolecule to the capture resin as is envisaged in the present invention may then result in reduced variation in the resulting biomolecule-effector-conjugate or biomolecule-reporter-conjugate produced by the above method. This would be advantageous. Another advantage of the resin systems of the present invention resides in the fact that a wider range of effector or reporter moieties can in principle be conjugated to the resin than is the case for conventional Protein A, Protein G or Protein L based systems. For example, in the case of hydrophobic molecules other non-specific binding that may occur in parent Protein A, Protein G or Protein L based systems may disrupt or prevent effective conjugation of such drugs.

In an embodiment, the capture resin is a non-proteinaceous capture resin. In an embodiment, the biomolecule capture moiety of the capture resin has a molecular weight of about 1000 Da or less, optionally about 500 Da or less, about 300 Da or less or about 200 Da or less. In an embodiment, the capture resin is a non-proteinaceous capture resin and the biomolecule capture moiety of the capture resin has a molecular weight of about 1000 Da or less. In a further embodiment, the capture resin is a non-peptide based capture resin and the biomolecule capture moiety of the capture resin has a molecular weight of about 1000 Da or less.

Another benefit of employing a non-peptide based Protein A, Protein G or Protein L mimetic as opposed to the employment of the parent Protein A, Protein G or Protein L or a peptide-based Protein A, Protein G or Protein L as the biomolecule capture moiety, is that the mimetic biomolecule capture moieties are compatible with a broad range of common antibody conjugation chemistries and can be scaled up to industrial levels. This is in contrast with Protein A, Protein G or Protein L based biomolecule capture moieties and peptide-based Protein A, Protein G or Protein L capture moieties.

For example, it is often desirable to target the lysyl side chain functional group on the immobilised antibody. Of the 28 antibody drug conjugates currently in clinical development almost half (those shaded grey in the table below) employ lysine directed conjugation chemistry. The proteinaceous nature of an immobilizing ligand on the surface of Protein A, G or L will result in the unintentional targeting of the lysyl side chain functional groups on the protein capture resin. Protein A (swiss-prot P02976) has 59 Lysine residues, Protein G (swiss-prot P919909) has 59 lysine residues and Protein L (swiss-prot Q51918) has 132 lysine residues.

In addition to the competition between ligand and antibody lysyl residues as described above, there are also other issues with Protein A, G and L based capture resins. These include leaching of the protein and immunogenicity of leached adducts. This means that these affinity supports cannot be employed (for purification or conjugation) towards the end of a manufacturing process. Any conjugate material furnished from such a process employing Protein A, G and L based capture resins will not meet current regulatory guidelines for antibody purification and product quality.

Processes for Synthesising a Biomolecule-Effector-Conjugate or Biomolecule-Reporter-Conjugate

In accordance with the present invention there are provided processes for synthesising a biomolecule-effector-conjugate or biomolecule-reporter-conjugate. The processes of the invention are depicted in the schematic below. The disclosure in the schematic below is not restrictive to the claims and content of the current invention but serves as a visual indictor to illustrative processes in which the invention may be used.

Method of Synthesising a Chemically or Enzymatically Modified or an Activated, Immobilised Biomolecule:

In accordance with the present invention there is provided a method of synthesising a chemically or enzymatically modified or an activated, immobilised biomolecule, the method comprising:

a) optionally contacting a biomolecule with a chemical modification agent, enzymatic modification agent or activating agent to provide a chemically modified, enzymatically modified or activated biomolecule;
b)
(i) when step (a) is carried out, contacting the chemically modified, enzymatically modified or activated biomolecule of step (a) with a capture resin comprising a non-peptide based Protein A, Protein G or Protein L mimetic biomolecule capture moiety under conditions suitable to immobilise the chemically modified, enzymatically modified or activated biomolecule and therefore provide an immobilised chemically modified, enzymatically modified or activated biomolecule; or
(ii) when step (a) is not carried out, contacting a biomolecule with a capture resin comprising a non-peptide based Protein A, Protein G or Protein L mimetic biomolecule capture moiety under conditions suitable to immobilise the biomolecule and therefore provide an immobilised biomolecule;
   c) contacting the immobilised chemically modified, enzymatically modified or activated biomolecule of step (b)(i) or the immobilised biomolecule of step (b)(ii) with a chemical modification agent, enzymatic modification agent or activating agent to provide an immobilised chemically modified, enzymatically modified and/or activated biomolecule;
   wherein the biomolecule is an antibody, modified antibody or antibody fragment.

Conjugation of proteins and more specifically antibodies is often used in research, diagnostics and therapeutics. Bioconjugate Techniques, Second Edition (Greg T. Hermanson) provides highly detailed information on the chemistry, reagent systems and practical applications for creating labelled or conjugate molecules. It also describes dozens of reactions with details on hundreds of commercially available reagents and the use of these reagents for modifying or crosslinking peptides and proteins, sugars and polysaccharides, nucleic acids and oligonucleotides, lipids, and synthetic polymers. A brief summary of key conjugation chemistries applied to antibodies is provided below.

Conjugation to free thiols after reduction of the native interchain disulphides is a common approach to antibody conjugation and the chemistry employed for the commercial antibody drug conjugate (ADC) ADCetris®. A process comprises contacting the antibody with a reductant such as TCEP, DTT, merceptoethylamine or other suitable reductant well known in the field followed by conjugation with a drug, ligand, label of the formula D-X, where D is the drug, ligand or label and X is a reactive group selected from maleimides, haloalkanes, pyridyl disulphides, enes, vinyl sulphones, bis-sulphones, acrylates, methacrylates and other thiol reactive chemistries known in the art.

An alternative approach to thiol conjugation with antibodies is to (genetically) engineer reactive cysteine residues at specific sites in antibodies to allow drugs, ligands or labels to be conjugated with defined stoichiometry without disruption of interchain disulphide bonds. The engineered cysteines are often present as mixed disulphides of cysteine or glutathione. The adducts are removed by complete reduction followed by diafiltration. This breaks the interchain disulphides which must be reformed by oxidation with air, CuSO₄ or dehydroascorbic acid.

Another common site for conjugation are amino groups present on the side-chain of lysine residues. The simplest approach comprises contacting the antibody with a drug, ligand, label or linker of the formula D-Y. D has the same definition as above and Y is a reactive group selected from isocyanates, NHS esters, sulfonyl chlorides, epoxides and other reagents known to those skilled in the art.

Indirect conjugation to lysines is often also employed. The amino group of the lysine side chain is first activated with a heterobifunctional linker before this is conjugated with a drug, ligand or label containing a complimentary reactive chemistry. Examples of such couplets include modification of the lysine with 2-iminothiolane to create a new thiol followed by conjugation with any of the thiol reactive drug-linkers (D-X) described above. Another couplet is the modification of lysine with the heterobifunctional crosslinker SMCC to create a lysine bound maleimide followed by conjugation with a drug, ligand or label containing a free thiol. For a complete review of potential couplets useful for indirect lysine conjugation see Hermanson and the Pierce/Thermo Scientific cross-linking agent catalogue.

Several groups have developed ways to incorporate non-natural amino acids with side chains that are chemically orthogonal to the 20 proteogenic amino acid side chains in proteins.

Redwood Bioscience (www.redwoodbioscience.com) has developed a technology they call Aldehyde Tagging. In this they exploit a natural enzyme called formyl glycine enzyme (FGE) which normally converts a Cys residue within a highly conserved 13 amino acid sequence into a formyl glycine (aldehyde) in Type I sulfatases (Wu et al, PNAS, 2009, 106, 9, 3001). Drugs, ligands or labels to be conjugated to such modified antibodies must contain aldehyde reactive chemistries such as oxyamines or hydrazines. A full disclosure of aldehyde reactive functionalities can be found in Hermanson and Perbio catalogues.

Ambryx has developed a technology they call EuCode (Liu et al, Anu. Rev. Biochem., 2010, 79, 413). EuCode is a platform whereby cells are engineered to incorporate non-natural amino acids in heterologous proteins by inclusion of three non-natural components in the expression system:

1. A non-natural amino acid supplemented into the medium
2. An orthogonal aminoacyl-tRNA synthetases (aaRS)
3. An orthogonal tRNA

The orthogonal aaRS/tRNA pair has been engineered/selected to promote read through at the amber stop codon and to incorporate the non-natural amino acid at that position. As many as 70 nnAAs have been incorporated into protein using this approach. The figure below expands on the possible combination of orthogonal amino acid side chain and reactive chemistry (adapted from Ambryx presentation at Hanson Wade ADC summit meeting in February 2012).

Sutro has described the production of antibodies and cytokines using an open, cell-free synthesis (OCFS) technology. A feature of OCFS is the ability to incorporate non-natural amino acids into the protein with charged tRNAs that can be directed to a specific codon to deliver the non-natural amino acid to a specific location on the protein—making the protein amenable to specific modification or imparting a new desired property (Goerke et al, Biotechnol. Bioeng., 2008, 99: 351-367).

Immobilized antibody conjugation is compatible with all non-natural amino acid side chains and complimentary reactive chemistries with one proviso. The antibody capture ligand must not contain the novel chemistry incorporated as part of the non-natural amino acid side chain.

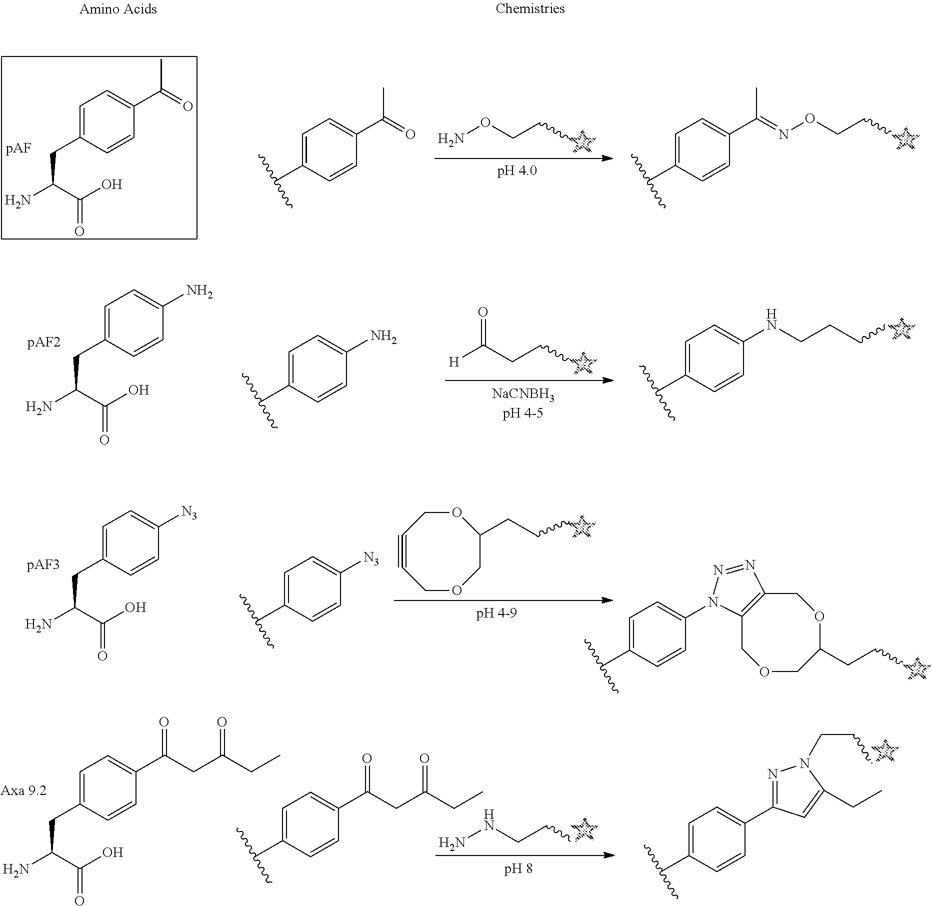

Oxidation of polysaccharide residues in glycoproteins with sodium periodate provides a mild and efficient way of generating reactive aldehyde groups for subsequent conjugation with amine or hydrazide containing molecules; drugs, ligands or labels. The process involve first contacting the antibody with sodium periodate and then conjugating with reactive groups selected from amines, hydrazides, aminoxy or other aldehyde reactive chemistries known in the art. The conjugation step is typically performed under acidic conditions to form oxime & hydrazone bonds. In a related approach Hydrazino-iso-Pictet-Spengler (HIPS) ligation also conjugates reactive aldehyde groups with substituted hydrazines to form stable azacarboline conjugates.

Step (a):

In an embodiment, step (a) is carried out.

In an alternative embodiment, step (a) is omitted.

In embodiment, the step of contacting a biomolecule with a chemical modification agent, enzymatic modification agent or an activating agent to provide a modified or activated, biomolecule involves reducing the biomolecule. In an embodiment, the reduction of the biomolecule involves complete reduction. In an embodiment, the reduction of the biomolecule involves partial reduction. In an embodiment, the reduction of the biomolecule involves complete reduction followed by re-oxidation.

In an embodiment, the biomolecule is reduced by contacting it with a reducing agent such as tris(2-carboxyethyl)phosphine (TCEP), dithiothreitol (DTT), merceptoethylamine or other suitable reductant. Preferably the reducing agent is tris(2-carboxyethyl)phosphine (TCEP).

In an embodiment, the reduced biomolecule is re-oxidised by contacting it with an oxidising agent such as air, CuSO₄ or dehydroascorbic acid (DHAA). Preferably the oxidising agent is dehydroascorbic acid (DHAA).

In an embodiment, the process of reducing the biomolecule is carried out in a buffer solution such as phosphate buffered saline (PBS).

In an embodiment, the process of reducing the biomolecule is carried out at a pH of from about 5 to about 10, preferably from about 7 to about 8, preferably about 7.4.

In an embodiment, the process of reducing the biomolecule is carried out in the presence of a chelating agent, such as EDTA.

In an embodiment, the process of reducing the biomolecule involves incubating the biomolecule with the reducing agent for a period of time of from about 20 minutes to about 3 days, optionally, from about 1 hour to about 2 days and further optionally from about 6 hours to about 18 hours.

In an embodiment, the step of contacting the biomolecule with a chemical modification agent, enzymatic modification agent or an activating agent to provide a modified or activated biomolecule involves reacting the biomolecule with a crosslinker moiety. For example, the crosslinker moiety could be an amine-to-sulfhydryl crosslinker, e.g. a crosslinker having an NHS-ester and a maleimide reactive group at opposite ends. This method of modifying or activating the biomolecule effectively results in a biomolecule-linker-effector-conjugate or a biomolecule-linker-reporter-conjugate. Suitable cross-linkers are generally able to react with a primary amine group on the effector or reporter group (via the reactive NHS ester end) and also react with a cysteine residue on the biomolecule (via the reactive maleimide end). In this particular example, the maleimide end will react with a cysteine in the immobilised biomolecule. An example of such a crosslinker is succinimidyl 4-(N-maleimidomethyl)cyclohexane-1-carboxylate (SMCC).

In an embodiment, the process of reacting with a crosslinker is carried out in a buffer solution such as phosphate buffered saline (PBS). Alternatively, the process of reacting with a crosslinker is carried out in a ‘Modification Buffer’ including a sodium phosphate buffer, NaCl and a chelating agent, such as EDTA.

In an embodiment, the process of reacting with a crosslinker is carried out at a pH of from about 7 to about 9, preferably from about 7 to about 8, preferably about 8.0.

In an embodiment, the process of reacting with a crosslinker is carried out in the presence of a chelating agent, such as EDTA.

In an embodiment, the process of reacting with a crosslinker involves incubating the biomolecule with the crosslinker for a period of time of from about 20 minutes to about 3 days, optionally, from about 1 hour to about 2 days and further optionally from about 6 hours to about 18 hours.

In an embodiment, the step of contacting the biomolecule with a chemical modification agent or an activating agent to provide a modified or activated biomolecule involves reacting the biomolecule with Traut's reagent.

In an embodiment, the process of reacting with Traut's reagent is carried out in a buffer solution such as phosphate buffered saline (PBS).

In an embodiment, the process of reacting with Traut's reagent is carried out at a pH of from about 7 to about 9, preferably from about 7 to about 8, preferably about 8.0.

In an embodiment, the process of reacting with Traut's reagent is carried out in the presence of a chelating agent, such as EDTA.

In an embodiment, the process of reacting with Traut's reagent involves incubating the biomolecule with the reducing agent for a period of time of from about 20 minutes to about 3 days, optionally, from about 1 hour to about 2 days and further optionally from about 6 hours to about 18 hours.

In an embodiment, the activated biomolecule is washed to remove any modification/activating agent. In an embodiment the washing involves rinsing with a buffer, optionally wherein the buffer is phosphate buffered saline (PBS). Other suitable buffers include: Potassium phosphate buffer; Sodium phosphate buffer; Sodium citrate buffer; Bis-Tris propane buffer; HEPES buffer; Sodium acetate buffer; Sodium citrate buffer; Cacodylic acid buffer; Ammonium acetate buffer; Imidazole buffer; Bicine buffer; and 2-(N-morpholino)ethanesulfonic acid (MES) buffer. For example, the biomolecule can be washed with a buffer solution such as phosphate buffered saline (PBS) at a pH of from about 7 to about 8, preferably about 7.4. Optionally, the rinsing of the activated biomolecule is carried out in the presence of a chelating agent, such as EDTA. Another example of rinsing the activated biomolecule involves rinsing the resin with a buffer such as PBS followed by a ‘Conjugation Buffer’ which includes sodium citrate, NaCl and a chelating agent such as EDTA.

Step (b):

When step (a) is carried out, step (b) involves contacting the chemically modified, enzymatically modified or activated biomolecule of step (a) with a capture resin comprising a non-peptide based Protein A, Protein G or Protein L mimetic biomolecule capture moiety under conditions suitable to immobilise the chemically modified, enzymatically modified or activated biomolecule and therefore provide an immobilised chemically modified, enzymatically modified or activated biomolecule.

When step (a) is omitted, step (b) involves contacting a biomolecule with a capture resin comprising a non-peptide based Protein A, Protein G or Protein L mimetic biomolecule capture moiety under conditions suitable to immobilise the biomolecule and therefore provide an immobilised biomolecule.

In an embodiment, the step of contacting the biomolecule with the capture resin comprises incubating the biomolecule with the capture resin.

The incubation may be carried out at temperature of from about 0° C. to about 100° C., preferably at temperature of from about 5° C. to about 50° C. and optionally at temperature of from about 10° C. to about 40° C. Ideally, the incubation is carried out at temperature of from about 15° C. to about 37° C., e.g. the incubation is carried out at room temperature, such as about 21° C. Alternatively, the incubation is carried out at about 37° C.

The incubation may be carried out for a period of time of from about 1 minute to about 3 days, e.g. for a period of time of from about 10 minutes to about 18 hours. Preferably the incubation is carried out for a period of time of from about 20 minutes to about 1 hour.

In an embodiment, the incubation is carried out in an aqueous media. In an alternate embodiment, the incubation is carried out in a buffer solution such as phosphate buffered saline (PBS) or any buffering salt compatible with the desired binding pH and chemistry, optionally the incubation is carried out in a buffer solution such as phosphate buffered saline (PBS). In an embodiment, the incubation is carried out using a co-solvent including a solvent such as DMSO, DMA or DMF. The co-solvent may be present within a range of 0.5-80% v/v, such as 0.5-50% v/v.

In an embodiment, the incubation is carried out at a pH of from about 5 to about 10, preferably about 5 to about 8, more preferably about 6 to about 8 In a preferred embodiment, the incubation is carried out at a pH of about 6 to about 7.5, ideally at pH of about 6.5. In another preferred embodiment, the incubation is carried out at a pH of about 7 to about 8, ideally at pH of about 7.4. This results in improved binding of the antibody to the derivatised support.

In an embodiment, the immobilised biomolecule (i.e. the biomolecule that is immobilised on the capture resin) is washed to remove any biomolecule that has not been immobilised on the capture resin. The washing of the immobilised biomolecule can be affected by rinsing with fresh solvent. For example, the washing of the immobilised biomolecule can be affected by rinsing with a buffer solution such as PBS. Optionally, the rinsing of the immobilised biomolecule is carried out in the presence of a chelating agent, such as EDTA. Alternatively, the washing of the immobilised biomolecule can be affected by rinsing with a ‘Modification Buffer’ including a sodium phosphate buffer, NaCl and a chelating agent, such as EDTA.

Step (c):

In an embodiment, step (c) is carried out.

In an alternative embodiment, step (c) is omitted.

Step (c) involves contacting the immobilised chemically modified, enzymatically modified or activated biomolecule of step (b)(i) or the immobilised biomolecule of step (b)(ii) with a chemical modification agent, enzymatic modification agent or activating agent to provide an immobilised chemically modified, enzymatically modified and/or activated biomolecule.

In an embodiment, the step of contacting the immobilised biomolecule with a chemical modification agent, enzymatic modification agent or an activating agent to provide a modified and/or activated, immobilised biomolecule involves reducing the biomolecule. In an embodiment, the reduction of the biomolecule involves complete reduction. In an embodiment, the reduction of the biomolecule involves partial reduction. In an embodiment, the reduction of the biomolecule involves complete reduction followed by re-oxidation.

In an embodiment, the biomolecule is reduced by contacting it with a reducing agent such as tris(2-carboxyethyl)phosphine (TCEP), dithiothreitol (DTT), merceptoethylamine or other suitable reductant. Preferably the reducing agent is tris(2-carboxyethyl)phosphine (TCEP).

In an embodiment, the reduced biomolecule is re-oxidised by contacting it with an oxidising agent such as air, CuSO₄ or dehydroascorbic acid (DHAA). Preferably the oxidising agent is dehydroascorbic acid (DHAA).

In an embodiment, the process of reducing the biomolecule is carried out in a buffer solution such as phosphate buffered saline (PBS).

In an embodiment, the process of reducing the biomolecule is carried out at a pH of from about 5 to about 10, preferably from about 7 to about 8, preferably about 7.4.

In an embodiment, the process of reducing the biomolecule is carried out in the presence of a chelating agent, such as EDTA.

In an embodiment, the process of reducing the biomolecule involves incubating the biomolecule with the reducing agent for a period of time of from about 20 minutes to about 3 days, optionally, from about 1 hour to about 2 days and further optionally from about 6 hours to about 18 hours.

In an embodiment, the step of contacting the immobilised biomolecule with a chemical modification agent, enzymatic modification agent or an activating agent to provide a modified or activated, immobilised biomolecule involves reacting the biomolecule with a crosslinker moiety. For example, the crosslinker moiety could be an amine-to-sulfhydryl crosslinker, e.g. a crosslinker having an NHS-ester and a maleimide reactive group at opposite ends. This method of modifying or activating the biomolecule effectively results in a biomolecule-linker-effector-conjugate or a biomolecule-linker-reporter-conjugate. Suitable cross-linkers are generally able to react with a primary amine group on the effector or reporter group (via the reactive NHS ester end) and also react with a cysteine residue on the biomolecule (via the reactive maleimide end). In this particular example, the maleimide end will react with a cysteine in the immobilised biomolecule. An example of such a crosslinker is succinimidyl 4-(N-maleimidomethyl)cyclohexane-1-carboxylate (SMCC).

In an embodiment, the process of reacting with a crosslinker is carried out in a buffer solution such as phosphate buffered saline (PBS). Alternatively, the process of reacting with a crosslinker is carried out in a ‘Modification Buffer’ including a sodium phosphate buffer, NaCl and a chelating agent, such as EDTA.

In an embodiment, the process of reacting with a crosslinker is carried out at a pH of from about 7 to about 9, preferably from about 7 to about 8, preferably about 8.0.

In an embodiment, the process of reacting with a crosslinker is carried out in the presence of a chelating agent, such as EDTA.

In an embodiment, the process of reacting with a crosslinker involves incubating the biomolecule with the crosslinker for a period of time of from about 20 minutes to about 3 days, optionally, from about 1 hour to about 2 days and further optionally from about 6 hours to about 18 hours.

In an embodiment, the step of contacting the immobilised biomolecule with a chemical modification agent or an activating agent to provide a modified or activated, immobilised biomolecule involves reacting the biomolecule with Traut's reagent.

In an embodiment, the process of reacting with Traut's reagent is carried out in a buffer solution such as phosphate buffered saline (PBS).

In an embodiment, the process of reacting with Traut's reagent is carried out at a pH of from about 7 to about 9, preferably from about 7 to about 8, preferably about 8.0.

In an embodiment, the process of reacting with Traut's reagent is carried out in the presence of a chelating agent, such as EDTA.

In an embodiment, the process of reacting with Traut's reagent involves incubating the biomolecule with the reducing agent for a period of time of from about 20 minutes to about 3 days, optionally, from about 1 hour to about 2 days and further optionally from about 6 hours to about 18 hours.

Step (d):

In an embodiment, step (d) is carried out.

In an alternative embodiment, step (d) is omitted.

In an embodiment, the immobilised chemically modified, enzymatically modified or activated biomolecule of step (b)(i); the immobilised biomolecule of step (b)(ii); or the immobilised chemically modified, enzymatically modified and/or activated, immobilised biomolecule of step (c) is washed to remove any modification/activating agent. In an embodiment the washing involves rinsing with a buffer, optionally wherein the buffer is phosphate buffered saline (PBS). Other suitable buffers include: Potassium phosphate buffer; Sodium phosphate buffer; Sodium citrate buffer; Bis-Tris propane buffer; HEPES buffer; Sodium acetate buffer; Sodium citrate buffer; Cacodylic acid buffer; Ammonium acetate buffer; Imidazole buffer; Bicine buffer; and 2-(N-morpholino)ethanesulfonic acid (MES) buffer. For example, the immobilised biomolecule can be washed with a buffer solution such as phosphate buffered saline (PBS) at a pH of from about 7 to about 8, preferably about 7.4. Optionally, the rinsing of the activated, immobilised biomolecule is carried out in the presence of a chelating agent, such as EDTA. Another example of rinsing the activated, immobilised biomolecule involves rinsing the resin with a buffer such as PBS followed by a ‘Conjugation Buffer’ which includes sodium citrate, NaCl and a chelating agent such as EDTA.

Step (e):

In an embodiment, step (c) is repeated once, twice or three times. In an embodiment, step (c) is repeated once. In an embodiment, step (c) is repeated twice. In an embodiment, step (c) is repeated three times.

In an embodiment, step (d) is repeated once, twice or three times. In an embodiment, step (d) is repeated once. In an embodiment, step (d) is repeated twice. In an embodiment, step (d) is repeated three times.

In an embodiment, step (c) is not repeated.

In an embodiment, step (d) is not repeated.

Step (f):

In an embodiment, step (f) is carried out.

In an alternative embodiment, step (f) is omitted.

Step (f) involves contacting an effector component or reporter component with a chemical modification agent, enzymatic modification agent or activating agent to provide a chemically modified, enzymatically modified and/or activated effector component or reporter component.

In an embodiment, the step of contacting the effector component or reporter component with a chemical modification agent, enzymatic modification agent or an activating agent to provide a modified or activated effector component or reporter component involves reacting the effector component or reporter component with a crosslinker moiety. For example, the crosslinker moiety could be an amine-to-sulfhydryl crosslinker, e.g. a crosslinker having an NHS-ester and a maleimide reactive group at opposite ends. This method of modifying or activating the effector component or reporter component effectively results in a biomolecule-linker-effector-conjugate or a biomolecule-linker-reporter-conjugate. Suitable cross-linkers are generally able to react with a cysteine residue on the biomolecule, e.g. the chemically modified, enzymatically modified or activated biomolecule, (via the reactive maleimide end) and also react with an amine moiety on the effector component or reporter component (via the reactive NHS ester end). In this particular example, the maleimide end will react with a cysteine in the immobilised biomolecule. An example of such a crosslinker is succinimidyl 4-(N-maleimidomethyl)cyclohexane-1-carboxylate (SMCC).

In an embodiment, the process of reacting with a crosslinker is carried out in a buffer solution such as phosphate buffered saline (PBS). Alternatively, the process of reacting with a crosslinker is carried out in a ‘Modification Buffer’ including a sodium phosphate buffer, NaCl and a chelating agent, such as EDTA.

In an embodiment, the process of reacting with a crosslinker is carried out at a pH of from about 7 to about 9, preferably from about 7 to about 8, preferably about 8.0.

In an embodiment, the process of reacting with a crosslinker is carried out in the presence of a chelating agent, such as EDTA.

In an embodiment, the process of reacting with a crosslinker involves incubating the effector component or reporter component with the crosslinker for a period of time of from about 20 minutes to about 3 days, optionally, from about 1 hour to about 2 days and further optionally from about 6 hours to about 18 hours.

Step (g):

Step (g) involves contacting the immobilised biomolecule or the immobilised chemically modified, enzymatically modified and/or activated biomolecule with the chemically modified, enzymatically modified or activated effector/reporter component of step (f) (when step (f) is carried out) or contacting the immobilised biomolecule or the immobilised chemically modified, enzymatically modified and/or activated biomolecule with an effector/reporter component to form an immobilised biomolecule-effector/reporter-conjugate.

In an embodiment, the step of contacting the immobilised biomolecule or the chemically modified, enzymatically modified and/or activated, immobilised biomolecule with the chemically modified, enzymatically modified or activated effector/reporter component of step (f) (when step (f) is carried out) involves simultaneously (1) carrying out the chemical modification, enzymatic modification or activation of the effector/reporter component and (2) contacting with the immobilised biomolecule or the chemically modified, enzymatically modified and/or activated, immobilised biomolecule. In other words, the biomolecule is contacted with the chemically modified, enzymatically modified or activated effector/reporter component as it is generated in situ. In this embodiment, steps (f) and (g) are not separate steps, but are a single, combined step.

In an embodiment, the step of contacting the immobilised biomolecule or the chemically modified, enzymatically modified and/or activated, immobilised biomolecule with an effector component to form an immobilised biomolecule-effector-conjugate involves contacting the chemically modified, enzymatically modified and/or activated, immobilised biomolecule with an effector component in a buffer solution as herein before described with relation to step (c).

In an embodiment, the step of contacting the immobilised biomolecule or the chemically modified, enzymatically modified and/or activated, immobilised biomolecule with an effector component to form an immobilised biomolecule-effector-conjugate involves contacting the chemically modified, enzymatically modified or activated, immobilised biomolecule with an effector component at a pH of from about 5 to about 8, preferably about 7 to about 8 and more preferably about 7.4.

In an embodiment, the step of contacting the immobilised biomolecule or the chemically modified, enzymatically modified and/or activated, immobilised biomolecule with an effector component to form an immobilised biomolecule-effector-conjugate is carried out in the presence of a chelating agent, such as EDTA.

In an embodiment, step of contacting the immobilised biomolecule or the chemically modified, enzymatically modified and/or activated, immobilised biomolecule with an effector component to form an immobilised biomolecule-effector-conjugate involves incubating the chemically modified, enzymatically modified or activated, immobilised biomolecule with effector component for a period of time of from about 20 minutes to about 3 days, optionally, from about 1 hour to about 2 days and further optionally from about 6 hours to about 18 hours.

In an embodiment, the step of contacting the immobilised biomolecule or the chemically modified, enzymatically modified and/or activated, immobilised biomolecule with a reporter component to form an immobilised biomolecule-reporter-conjugate involves contacting the chemically modified, enzymatically modified and/or activated, immobilised biomolecule with a reporter component in a buffer solution as herein before described with relation to step (c).

In an embodiment, the step of contacting the immobilised biomolecule or the chemically modified, enzymatically modified and/or activated, immobilised biomolecule with a reporter component to form an immobilised biomolecule-reporter-conjugate involves contacting the chemically modified, enzymatically modified or activated, immobilised biomolecule with a reporter component at a pH of from about 5 to about 8, preferably about 7 to about 8 and more preferably about 7.4.

In an embodiment, the step of contacting the immobilised biomolecule or the chemically modified, enzymatically modified and/or activated, immobilised biomolecule with a reporter component to form an immobilised biomolecule-reporter-conjugate is carried out in the presence of a chelating agent, such as EDTA.

In an embodiment, step of contacting the immobilised biomolecule or the chemically modified, enzymatically modified and/or activated, immobilised biomolecule with a reporter component to form an immobilised biomolecule-reporter-conjugate involves incubating the chemically modified, enzymatically modified or activated, immobilised biomolecule with reporter component for a period of time of from about 20 minutes to about 3 days, optionally, from about 1 hour to about 2 days and further optionally from about 6 hours to about 18 hours.

Step (h):

In an embodiment, step (h) is carried out.

In an alternative embodiment, step (h) is omitted.

In an embodiment, the immobilised conjugate is washed prior to the step of releasing purified conjugate from the capture resin. The washing removes any unreacted effector/reporter component. In an embodiment the washing involves rinsing with a buffer, optionally wherein the buffer is phosphate buffered saline (PBS), and other solvent. Other suitable buffers include: Potassium phosphate buffer; Sodium phosphate buffer; Sodium citrate buffer; Bis-Tris propane buffer; HEPES buffer; Sodium acetate buffer; Sodium citrate buffer; Cacodylic acid buffer; Ammonium acetate buffer; Imidazole buffer; Bicine buffer; and 2-(N-morpholino)ethanesulfonic acid (MES) buffer. For example, the immobilised conjugate can be washed with a buffer solution such as phosphate buffered saline (PBS) and dimethylacetamide (DMA) at a pH of from about 5 to about 7. Optionally, the rinsing of the immobilised conjugate is carried out in the presence of a chelating agent, such as EDTA.

In an embodiment, the immobilised conjugate is washed prior to the step of releasing the purified conjugate from the capture resin with a buffer, optionally wherein the buffer is phosphate buffered saline (PBS) or other buffer suitable for formulation. The washing removes any residual or superfluous organic solvent such as DMSO, DMA or DMF.

Step (i):

In an embodiment, the step of releasing the immobilised biomolecule-effector-conjugate or biomolecule-reporter-conjugate from the capture resin involves:

a) exposing the support-biomolecule compound to a release agent; and/or
b) altering the pH to break the support-biomolecule bond.

In an embodiment, the release agent is a hydrogen bond disrupter such as co-solvents of hexafluoroisopropanol, 2,2,2-trifluoroethanol or dimethylsulfoxide (DMSO).

In an embodiment, the release agent is incubated with the support-biomolecule.

The incubation may be carried out at temperature of from about 0° C. to about 100° C., preferably at temperature of from about 5° C. to about 50° C. and optionally at temperature of from about 10° C. to about 40° C. Ideally, the incubation is carried out at temperature of from about 15° C. to about 37° C., e.g. the incubation is carried out at room temperature, such as about 21° C. Alternatively, the incubation is carried out at about 37° C.

The incubation may be carried out for a period of time of from about 1 minute to about 3 days. Preferably the incubation is carried out for a period of time of from about 30 minutes to about 2 hours.

The incubation may be carried out in an aqueous media. Alternatively, the incubation may be carried out in a solvent such as DMF, DMA, DMSO, MeOH or MeCN. Alternatively, the incubation may be carried out in an aqueous-solvent mixture with up to 80% solvent, preferably 0.5% to 50% and most preferred 0.5% to 10% v/v. In certain cases mixtures of one or more of the above solvents, including water, may be appropriate. Where necessary a stabiliser may also be included to ensure the conjugate remains intact.

In an embodiment, the step of releasing the biomolecule-effector-conjugate or biomolecule-reporter-conjugate from the capture resin involves altering the pH. The pH can be altered by any amount that is sufficient to break the support-biomolecule bond but which will not affect the activity, integrity or 3D structure of the biomolecule.

For example, the pH can be adjusted so that it is acidic. In an embodiment, the pH is decreased from about pH2 to about pH6. Optionally, the pH is adjusted to be less than about pH 5, e.g. about pH 3 to about 5, for example less than about pH 4. In an embodiment, the pH is decreased to about pH 3.

Alternatively, the pH can be adjusted so that it is basic. In an embodiment, the pH is increased to about pH8 to about pH10. Optionally, the pH is adjusted to greater than pH 8. For example, the pH can be increased to about pH 9. The pH can be increased to being greater than pH 9. For example, the pH can be increased to about pH10. The pH can be increased to being greater than pH10, but usually will be less than pH14.

The biomolecule-effector-conjugate or biomolecule-reporter-conjugate may undergo one or more treatments with release agent. Advantageously, the use of a second or subsequent treatment with fresh release agent may result in increasing the amount of biomolecule-effector-conjugate or biomolecule-reporter-conjugate released from the capture resin. Fresh release agent is release agent that has not previously been incubated with the biomolecule-effector-conjugate or biomolecule-reporter-conjugate.

In an embodiment, the step of releasing the biomolecule-effector-conjugate or biomolecule-reporter-conjugate from the capture resin involves contacting the biomolecule-effector-conjugate or biomolecule-reporter-conjugate with a salt. For example, the biomolecule-effector-conjugate or biomolecule-reporter-conjugate might be contacted with NaCl. The concentration of the salt can range from about 0.1M to about 10M, preferably about 0.1M to about 1M.

In an embodiment, the eluted biomolecule-effector-conjugates or biomolecule-reporter-conjugates is neutralised after the step of releasing the conjugate from the capture resin. For example, the conjugate can be captured into 2% v/v of 1M tris(hydroxymethyl)aminoethane (TRIS).

Washing Steps:

In an embodiment the step of washing an intermediate in the method of the invention comprises removing substances that are not bound to the capture resin such as contaminants. Typical contaminants include excess reagent used to activate the immobilised biomolecule, biomolecule that has not been immobilised on the capture resin, effector component that has not reacted with the activated, reporter component that has not reacted with the activated, immobilised biomolecule or superfluous residual solvent or co-solvent. Any medium that does not affect the activity, integrity or 3D structure of the biomolecule or the integrity of the binding between the immobilised biomolecule and the capture resin can be used to wash the intermediate.

Preferably the buffer is isotonic and induces a stable environment to biomolecules such as antibodies by mimicking physiological pH and ionic strength. In an embodiment, the activated, immobilised biomolecule is washed by filtration. Optionally, the resultant filtrate is buffer-exchanged, e.g. by centrifugation using membrane cartridges.

Typically, additives are introduced to the buffer media. These additives induce a level of control to the buffer system and the biomolecule contained within it. For example, additives such as Tris or histidine are introduced to a buffered process stream to maintain pH and minimise incidental acidification. Typically, the pH of a biomolecule process stream should be maintained between pH5 and 9.5, with the extremes of the pH limits avoided for prolonged periods. Inorganic salts such as 0.1M NaCl(aq.) may be added to maintain the ionic strength of the process stream. Ionic and non-ionic detergents such as Tween (polysorbate) may be added to the buffer to favourably increase the solubility of poorly soluble biomolecules in the buffer media and minimise aggregation.

A Mixture Comprising a Capture Resin and an Activating Agent:

In accordance with the present invention there is provided a mixture comprising:

(i) a capture resin comprising an antibody, modified antibody or antibody fragment capture moiety selected from the group consisting of: a non-peptide-based Protein A, Protein G or Protein L mimetic, and
(ii) a chemical modification agent or activating agent.

In an embodiment, the capture resin includes an immobilised antibody, modified antibody or antibody fragment on the surface thereof.

A Use of a Capture Resin in the Synthesis of a Biomolecule-Effector-Conjugate:

In accordance with the present invention there is provided a use of a capture resin comprising an antibody, modified antibody or antibody fragment capture moiety selected from the group consisting of: a non-peptide-based Protein A, Protein G or Protein L mimetic, in the synthesis of a biomolecule-effector-conjugate.

A Use of a Capture Resin in the Synthesis of a Biomolecule-Reporter-Conjugate:

In accordance with the present invention there is provided a use of a capture resin comprising an antibody, modified antibody or antibody fragment capture moiety selected from the group consisting of: a non-peptide-based Protein A, Protein G or Protein L mimetic, in the synthesis of a biomolecule-reporter-conjugate.

Capture Resin:

For years researchers have tried to develop ligands that have affinity for a range of full length antibodies, fragments or fusions as replacements for traditional Protein A, G or L affinity purification supports. The main criterion for successful ligand discovery/development has been:

1. High selectivity for antibodies to afford high initial purification
2. Useful dynamic binding capacity
3. Elution conditions compatible with retention of antibody integrity
4. Stability of support during multiple elution/cleaning cycles
5. Lowered cost relative to Protein A, G or L supports

In the context of using these ligands for solid phase antibody conjugation criterion 1 above is not critical as the conjugation process starts with purified antibody. However, the ligand must meet the remaining 4 criterion in full. In addition, the ligand must ideally have a defined site of interaction with the antibody which affords suitable affinity binding strength for conjugation. This attribute is necessary so that the antibody may be bound to the support and not inadvertently eluted during buffer replenishment over time. In addition, a defined site of interaction is desirable to infer consistent conformational presentation of the bound antibody complex to the surrounding solution phase with the effect of providing a means for consistent and reproducible conjugation chemistry. Antibodies are well characterized biomolecules with a number of well-defined binding domains which are exploited for affinity purification.

The first defined region(s) are the Protein A and Protein G binding pockets which are exploited in affinity chromatography using Protein A/G and mimetics of Protein A/G supports. Protein A interacts with the CH2 CH3 interchain domain in the Fc region via number of non-covalent interactions with amino acid residues: Thr 250, Leu 251, Met 252, Ile 253, His 310, Gin 311, Leu 314, Asn 315, Lys 338, Glu 345, Ala 431, Leu 432, His 433, Asn 434 and His 435. Protein A mimetic supports have been rationally designed to interact with this domain via one or more of the amino acids defined above. These mimetic supports afford suitable affinity ligands for IgG binding and conjugation. Protein A mimetic supports may be defined in sub-classes as incorporating non-peptide, peptide or amino acid based ligands. Similarly, Protein G interacts with the CH2 CH3 interchain domain in the Fc region via number of non-covalent interactions with amino acid residues Ile 253, Ser 254, Gin 311, Glu 380, Glu 382, His 433, Asn 434 and His 435. Protein G mimetic supports have been rationally designed to interact with this domain via one or more of the amino acids described above. Once again these mimetic supports afford suitable affinity ligands for IgG binding and conjugation. Protein G mimetic supports may be defined in sub-classes as incorporating non-peptide, peptide or amino acid based ligands. In an embodiment, the capture resin is able to bind to a Protein A or a Protein G binding pocket on a biomolecule. A commercial embodiment of Protein A mimetics is Mabsorbent™ A1P, A2P and A3P (ProMetic Biosciences). These affinity supports meet the criterion for a Protein A mimetic as these non-peptide supports mimic the Phe-132, Tyr-133 dipeptide binding site in the hydrophobic core structure of Protein A.

A second defined region is the antibody light chain as targeted by a Protein L affinity matrix. Protein L binds specifically to Kappa I, II and IV light chains but not Kappa III nor Gamma light chains. The interaction between Protein L with antibodies has been mapped and it was noted that hydrogen bonds and salt bridges are important in binding. A total of 11 hydrophilic amino acid residues—namely; Ala, Asp, Gin, Glu, Gly, Ile, Leu, Lys, Phe, Thr, Tyr—of the Protein L domain are important in forming these bonds. Protein L mimetic affinity supports have been developed by creating triazine scaffold combinatorial libraries using structurally similar chemical compounds to the 11 amino acids disclosed above (WO 2004/035199A). Disclosed within WO2004/035199A a Protein L mimetic is defined as a ligand having 50% of the affinity of Protein L for an antibody or fragment and specificity for the light chain as evidenced by binding of Fab fragments. Any suitable scaffold disclosed herein or known to those skilled in the art can be substituted for the triazine scaffold as long as the characteristics of affinity and specificity for light chain are retained. Such resins are useful for the immobilization of antibodies and fragments containing Kappa I, II and IV light chains. One commercial embodiment of Protein L mimetics is Fabsorbent™ F1P HF (ProMetic Biosciences). This affinity support meets the criterion for a Protein L mimetic but also binds gamma light chain containing antibodies and fragments. Therefore, this affinity support is universally applicable to antibody affinity binding and conjugation. In an embodiment, the capture resin is able to bind to an antibody light chain as targeted by a Protein L affinity matrix.

A third defined region is the conserved nucleotide domain in the Fab arm of all antibody isotypes across a wide range of species. The binding site comprises 4 amino acid residues with the first being either a Tyr or Phe and the remaining three Tyr, Tyr and Trp. While the binding pocket location and amino acid side-chain orientation are conserved in the crystal structure overlay, there are slight differences in the overall backbone sequence variation from antibody to antibody and in numbering schemes. This is demonstrated below by comparing the conserved nucleotide binding sites for the commercial antibodies Herceptin and Rituximab. Nucleotide mimetics (non-peptide, peptide, nucleotide analogue and amino acid) which have been rationally designed to interact with this domain via one or more of the amino acids described above are suitable affinity ligands for IgG binding and conjugation.

| Antibody | Amino Acid 1 | Amino Acid 2 | Amino Acid 3 | Amino Acid 4 |
|---|---|---|---|---|
| Herceptin | Light Chain Tyr 36 | Light Chain Tyr 87 | Heavy Chain Tyr 95 | Heavy Chain Trp 110 |
| Rituximab | Light Chain Phe 35 | Light Chain Tyr 86 | Heavy Chain Tyr 95 | Heavy Chain Trp 111 |

In an embodiment, the capture resin is able to bind to a conserved nucleotide domain in the Fab arm of an antibody.

A fourth defined region is the glycan structures present on Asn 297 in the CH2 domain of the Fc region of intact antibodies. m-Aminophenylboronic acid acting as an affinity ligand binds to cis diol groups on sugar residues such as mannose, galactose or glucose such that are present with the saccharide moiety of glycoprotein molecules. A reversible five membered ring complex is furnished from this interaction. A typical antibody glycan structure is shown below to highlight the presence of mannose and galactose in antibody glycans (Adapted from Arnold et al, Advances in Experimental Medicine and Biology, 2005, 564, 27-43). In an embodiment, the capture resin is able to bind to a glycan structure present on Asn 297 in the CH2 domain of the Fc region of intact antibodies.

Ligands can be attached to a range of solid support matrices well known in the field of affinity chromatography. These include by example, synthetic polymers such as polyacrylamide, polyvinylalcohol or polystyrene, especially cross linked synthetic polymers, inorganic supports such as silica-based supports and in particular polysaccharide supports such as starch, cellulose and agarose.

Specific ligand-supports suitable for antibody binding are described below:

‘Non-Peptide’ Protein A, G and L mimetic affinity supports

Molecular modelling of the Protein A, G or L interaction combined with synthetic chemical library screening has enabled semi-rational design of small molecule mimetics of these proteins (Li et al, Nature Biotechnology, 1998, 16, 190-195). Examples of such resins include the commercially available supports mAbsorbent A1P, mAbsorbent A2P and FAbsorbent F1P HF (ProMetic Biosciences).

mAbsorbent A1P, mAbsorbent A2P HF and FAbsorbent F1P HF supports are formed on a synthetic aromatic triazine scaffold (www.prometicbioscience.com).

US20010045384 discloses a Protein A mimetic ligand-complex assembled upon an imino diacetate (IDA) type scaffold. The IDA scaffold is derivatised with triazyl ligands to afford a multivalent triazyl ligand-complex.

WO9808603 describes the isolation of immunoglobulins from cell culture supernatants, sera, plasma or colostrum using affinity resins. These affinity resins comprise of synthetic mono or bicyclic-aromatic or heteroaromatic ligands to facilitate immunoglobulin purification.

Another ligand with promise as an antibody affinity resin is sulfamethazine. Dextran microparticles coupled with sulfamethazine specifically bind antibodies (Yi et al, Prep. Biochem. Biotechnol., 2012, 42, 6, 598-610).

In the selection of the lead candidate ligands described above many ligands were excluded based on lack of antibody specificity. It is disclosed herein that specificity is less important than binding efficiency, capacity and stability for a solid phase antibody conjugation resin and as such these are not discounted.

‘Peptide’ Protein A, G or L Mimetic Affinity Supports

A number of Protein A mimetic peptides have been disclosed. Menegatti identified a hexapeptide with the sequence HWRGWV that binds to the antibody Fc region (Menegatti et al, Journal of Separation Science, 2012, 35, 22, 3139-3148. Fassina et al have identified a Protein A mimetic peptide TG191318 through synthesis and screening of synthetic multimeric peptide libraries composed of randomized synthetic molecules with a tetradendate lysine core (Fassina et al, J. Mol. Recognit., 1996, 9, 564). EP1997826 discloses a peptide comprising X₁-Arg-Thr-Tyr. Lund et al discloses two peptide ligands suitable for antibody affinity chromatography (Lund et al, J Chromatogr. A, 2012, 1225, 158-167). DAAG and D₂AAG contain L-arginine, L-glycine and a synthetic aromatic acid 2, 6-di-tert-butyl-4-hydroxybenzyl acrylate (DBHBA)

Amino Acid Protein A, G or L Mimetic Affinity Supports

In addition to the complex macromolecular ligands described above simple amino acids have been proposed as Protein A mimetics that bind antibodies in the same way (Naik et al, J. Chromatogr. A, 2011, 1218, 1756-1766). An example of this is AbSep a tryptophan containing polymethacrylate resin with a high affinity for the Protein A binding site in the Fc region of antibodies. Resins containing the amino acids Tyrosine, Histidine and Phenylalanine are also suitable for antibody immobilisation and conjugation (Bueno et al, J. Chromatogr. B, Biomed. Appl., 1995; 667, 1, 57-67).

Nucleotide Binding Site Affinity Supports

Another strategy for developing antibody purification ligands has exploited the lesser known conserved nucleotide binding site (NBS) in the Fab variable regions of antibodies (Alves et al, Anal. Chem., 2012, 84, 7721-7728). The nucleotide analogue indolebutyric acid has been coupled to a ToyoPearl AF-650-amino M resin to prepare a support which meets criterion 1-5 above. An extensive range of other nucleotide analogues useful for antibody affinity chromatography is described in WO/2012/099949.

Carbohydrate Binding Resins

The ligand m-aminophenylboronic acid immobilised on a variety of supports has been used to purify glycoproteins. The ligand binds to cis-diol groups on sugar residues such as mannose, galactose, or glucose that are present within the saccharide moiety of glycoprotein molecules including antibodies, forming a reversible five-member ring complex. This complex can be dissociated by lowering the pH, or by using an elution buffer containing either Tris or sorbitol.

A ligand of the capture resin is able to interact with a biomolecule by specific, reversible and non-covalent bond interactions between the ligand and the biomolecule, e.g. a protein, antibody, modified antibody or antibody fragment. Non-covalent interactions may be classified as ionic, van der Waals, hydrogen bond or hydrophobic. These interactions work in a 3-dimensional manner to assist in the flexibility and conformation of the target biomolecule to the ligand of the capture resin. When in close proximity to the ligand, the biomolecule may infer one, several or all of these interactions to afford a ligand-biomolecule complex. The distance between the ligand and the biomolecule and the polarity and electronegativity of the ligand will determine the intensity of these interactions. Furthermore, the intensity of these interactions may be defined as the affinity force. A high affinity force between a ligand and a biomolecule constitutes a ligand-biomolecule complex of enhanced stability (US2009/0240033).

In an embodiment the capture resin comprises a non-peptide-based Protein A, Protein G or Protein L mimetic. The capture resin is able to bind an antibody, modified antibody or antibody fragment.

Non-peptide-based Protein A, Protein G or Protein L mimetics have been used in dye ligand chromatography, which is a mode of affinity chromatography that utilizes covalently bond textile dyes immobilised to a solid support such as agarose to purify proteins. These dyes resemble natural substrates/protein ligands to which proteins have affinities for. This mode of purification and separation is often referred to as pseudo-affinity chromatography. Dye ligand affinity chromatography is non-specific but the technique is advantageous for a broad binding range for a variety of proteins. Advances in the purification technique employed modified dyes to act as competitive inhibitors for a proteins normal substrate/ligand (P. Dean et al, J. Chromatography, 1979, 165, 3, 301-319). Triazinyl based ligands such as Cibacron Blue 3GA, Procion Red H-3B, Procion Blue MX 3G, Procion Yellow H-A, etc. are commonly employed and address the concerns of purity, leakage and toxicity of the original commercial dyes such as Blue Dextran (Lowe et al, Trends Biotechnology, 1992, 10, 442-448). Triazinyl ligands have been successfully used for the purification of albumin, oxidoreductases, decarboxylases, glycolytic enzymes, nucleases, hydroloases, lyases, synthetases and transferases (N. Labrou, Methods Mol. Biol. 2002, 147, 129-139). A limitation of biomimetic dye ligand affinity chromatography is that the affinity strength from biomolecule to biomolecule is considerably variable and in many cases a ligand that affords strong affinity strength for a protein may not be applicable to another protein. Therefore, it is often a necessity that an extensive and empirical screening process is undertaken to identify suitable synthetic ligands with desired affinity for a biomolecule of interest.

Consequently to assist in the structured elucidation of suitable ligands that effect affinity binding to a biomolecule a multivalent scaffold motif has been incorporated into the ligand structure to provide a construct to which a library of ligands may be introduced and screened in combination with rigid spatial separation of the ligand from the support.

In an embodiment, the ligand of the capture resin has a structure according to the structures recited in the disclosure of WO98/08603. The capture resins of WO98/08603 comprise synthetic mono or bicyclic-aromatic or heteroaromatic ligands to facilitate immunoglobulin purification. The contents of WO98/08603 relating to the structure of the capture resin are incorporated herein by reference. WO98/08603 describes the isolation of immunoglobulins from cell culture supernatants, sera, plasma or colostrum using affinity resins.

In an embodiment, the ligand of the capture resin has a structure according to the structures recited in the disclosure of WO2009/141384. The capture resins of WO2009/141384 have the general formula:

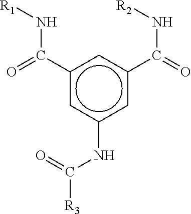

wherein R₁, R₂ and R₃ represent organic moieties of a molecular weight of 15-1000 g/mol, the total weight being 200-2000 g/mol, to which the ligand is immobilised to a solid phase support through an amide bond through one of R₁, R₂ and R₃. The contents of WO2009/141384 relating to the structure of the capture resin are incorporated herein by reference. WO2009/141384 describes that the ligands bind proteinaceous Factor VII polypeptides.

In an embodiment, the ligand of the capture resin has a structure according to the structures recited in the disclosure of US20010045384. The capture resins of US20010045384 are Protein A mimetic ligand-complexes assembled upon an imino diacetate (IDA) type scaffold. The contents of US20010045384 relating to the structure of the capture resin are incorporated herein by reference. The IDA scaffold is derivatatised with triazyl ligands to afford a multivalent triazyl ligand-complex. An illustrative triazyl ligand complex defined within US20010045384 is shown below:

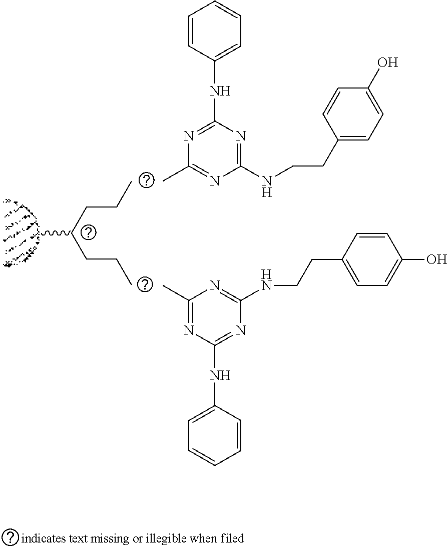

This Protein A mimetic has been demonstrated for utility as an affinity purification media for immunoglobulins such as IgG. It is postulated that the molecular geometry of the adjacent triazine ligands in the ligand-complex is an advantage using the IDA scaffold.

Another illustrative complex defined within US20010045384 is shown below:

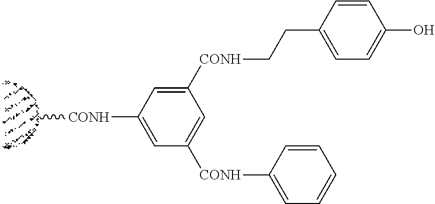

This branched multivalent phthalic acid-ligand scaffold Protein A mimetic ligand-complex was demonstrated to have affinity for immunoglobulins.

In an embodiment, the ligand of the capture resin has a structure according to the structures recited in the disclosure of WO9710887 and U.S. Pat. No. 6,117,996. The contents of WO9710887 and U.S. Pat. No. 6,117,996 relating to the structure of the capture resin are incorporated herein by reference. WO9710887 and U.S. Pat. No. 6,117,996 disclose a triazyl-ligand affinity construct of the type:

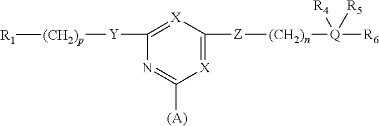

wherein, (A) represents the covalent attachment point of the triazine scaffold to a polysaccharide solid support optionally through a spacer arm interposed between the ligand and the solid support, and R₁ and Q are optionally substituted ligands with affinity for proteinaceous materials. The organic moieties are described as Protein A mimetics and are proposed and exemplified as alternative purification media to Protein A for the purification of proteinaceous materials.

In an embodiment, the ligand of the capture resin has a structure according to the structures recited in the disclosure of WO2004/035199. The content of WO2004/035199 relating to the structure of the capture resin is incorporated herein by reference. WO2004/035199 discloses the use of a Protein L mimetic comprising of a branched ligand scaffold of general formula,

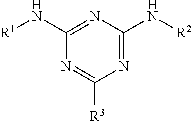

wherein R¹ and R² are the same or different and are each optionally substituted alkyl or aryl ligands, and R³ is a solid support optionally attached by a spacer motif. The triazyl-ligand scaffold has been disclosed as suitable Protein L mimetic ligands for the affinity binding of immunoglobulin or fragment antibodies (fAb) thereof. Furthermore, it is disclosed that these triazyl-ligand scaffolds have preferential binding affinity for immunoglobulin K and X light chains.

In an embodiment, the ligand of the capture resin has a structure according to the structures recited in the disclosure of US20110046353. The content of US20110046353 relating to the structure of the capture resin is incorporated herein by reference. US20110046353 discloses the purification of a fragment antibody (fAb) from a production medium. Fragment antibodies cannot be purified on Protein A media. The fAb is characterised as having a binding domain capable of binding to an antigen and in many embodiments disclosed within consists of having one heavy chain (Vh), or a functional fragment thereof, and one light chain (VI), or a functional fragment thereof, together with at least one other chain. Defined within are affinity ligands for fAb, consisting of a branched triazyl scaffold of the formula,

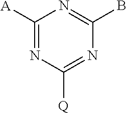

wherein Q represents the attachment point to a solid support matrix, optionally with a spacer motif and Groups A and B are phenyl or naphthyl groups substituted with one or more substituents capable of hydrogen bonding, preferably one or more of —OH, —SH or —CO₂H. Excellent results have been reported using supported affinity ligands commercially available from Prometic Biosciences under the trade names MAbsorbent A1P, MAbsorbent A2P & FAbsorbent F1P.

In an embodiment, the ligand of the capture resin has a structure:

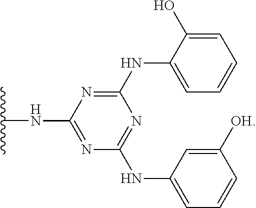 [SMILES: *Nc1nc(Nc2cccc(O)c2)nc(Nc2ccccc2O)n1]

In an embodiment, the ligand of the capture resin has a structure:

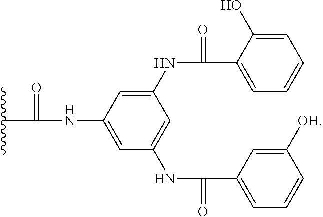 [SMILES: *C(=O)Nc1cc(NC(=O)c2cccc(O)c2)cc(NC(=O)c2ccccc2O)c1]

In an embodiment, the ligand of the capture resin has a structure:

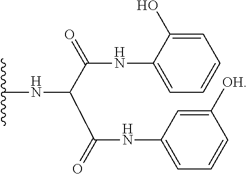 [SMILES: *NC(C(=O)Nc1cccc(O)c1)C(=O)Nc1ccccc1O]

In an embodiment, the capture resin is in the form of a bead. In an embodiment, the size of the bead in terms of the bead diameter is from about 10 μm to about 2000 μm, preferably from about 50 μm to about 1000 μm, and most preferably from about 75 μm to about 500 μm.

In an embodiment, the capture resin includes a mobile support made from a material selected from the group consisting of: Polystyrene, Polystyrene (PS-DVB)—Lightly cross-linked with divinylbenzene (0.1-5.0% DVB, termed Microporous), Polystyrene (PS-DVB) -Highly cross-linked with divinylbenzene (5-60% DVB, termed Macroporous), Polyethylene glycol, Polyethylene glycol grafted polystyrene (PS-PEG co-polymer), Poly acrylamide, Controlled Pore Glass (CPG) beads, Silica, Kieselguhr, Polypropylene, Poly(tetrafluoroethylene), Polyethylene, Cellulose, Poly methacrylate, Functionalised Monoliths, Functionalised Fibres, Monolithic columns (such as Nikzad et al, OPRD, 2007, 11, 458-462), Functionalised membranes, Agarose, Sepharose and Magnetic recoverable polymer beads.

In a preferred embodiment, the capture resin is a mobile support made from a material selected from the group consisting of: Agarose, Sepharose and Cellulose.

In an embodiment, the capture resin is a commercially available capture resin such as Fabsorbant™ F1P HF resin. In an embodiment, the capture resin is a commercially available capture resin such as Mabsorbant™ A1P or A2P resin.

Biomolecule:

In an embodiment, the biomolecule naturally occurs in a living organism. Alternatively, the biomolecule may be a derivative of a chemical compound that naturally occurs in a living organism. For example, the biomolecule may be biomolecule that has been altered chemically or genetically in a way which does not affects its biological activity.

In an embodiment, the biomolecule is an antibody.

In an embodiment, the biomolecule is a modified antibody, e.g. an antibody including a non-natural amino acid.

In an embodiment, the biomolecule is an antibody fragment.

In an embodiment, the antibody is a monoclonal antibody.

In an embodiment, the antibody is trastuzumab.

In an embodiment, the antibody, modified antibody or antibody fragment is an immunoglobulin (Ig), e.g. one of the five human immunoglobulin classes: IgG, IgA, IgM, IgD and IgE. The term antibody encompasses monoclonal antibodies. The term antibody encompasses polyclonal antibodies. The term antibody encompasses antibody fragments so long as they exhibit the desired biological activity. The antibody can be a human antibody, an animal antibody, a murine antibody, a humanised antibody or a chimeric antibody that comprises human and animal sequences.

The basic unit of the antibody structure is a heterotetrameric glycoprotein complex of at least 20,000 Daltons, for example about 150,000 Daltons. An antibody might be at least 900 amino acids in length, for example 1400 amino acids in length. An antibody may composed of two identical light (L) chains and two identical heavy (H) chains, linked together by both non-covalent associations and by di-sulfide bonds. Each heavy and light chain also has regularly spaced intrachain disulfide bridges. Each heavy chain is about 50,000 Daltons. Each heavy chain is at least 300 amino acids in length, for example about 450 amino acids in length. The antibody may be a heavy chain only antibody. Each light chain is about 20,000 Daltons. Each light chain is at least 100 amino acids in length, for example about 250 amino acids in length.

An antibody biomolecule can contain two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell).

In an embodiment the biomolecule is an antibody fragment. Antibody fragments comprise a portion of a full length antibody, generally the antigen binding or variable region thereof.

Examples of antibody fragments include Fab, pFc′, F(ab′)2, and scFv fragments; dsFv, diabodies; affibodies; minibodies; linear antibodies; single-chain antibody biomolecules including nanobodies and variable new antigen receptor (VNARs) and multispecific antibodies formed from antibody fragments. An antibody fragment might be at least 10 amino acids in length, for example an antibody fragment might be at least 20, 40, 60, 80, 100, 120, 140, 160, 180, 200, 220, 240, 260, 280 or 300 amino acids in length.

In an embodiment the biomolecule is a modified antibody or a modified antibody fragment. By “modified antibody” or “modified antibody fragment” is meant an antibody that differs from a parent antibody by virtue of at least one amino acid modification. A modified antibody or modified antibody fragment is an antibody or antibody fragment that has been previously chemically or enzymatically modified or genetically engineered (for example, to include a non-natural amino acid, etc) prior to being subjected to a method of the present invention.

A modified antibody or modified antibody fragment refers to an antibody, which in comparison to the wild-type antibody, is different with respect to its size, or which is different with respect to its glycosylation but which has a similar affinity to its ligand as the wild-type antibody.

Reporter Group

The term ‘reporter group’ in the conjugates according to the present invention includes any group or compound which is easily detectable by analytical means in vitro and/or in vivo and which confers this property to the conjugate. A reporter compound may confer properties to the conjugate not naturally occurring in the biomolecule.

In an embodiment, the reporter group is a label. Labels include modifying agents and compounds used to label biomolecules. These compounds may contain functional groups that provide and infer sensitive detectability measurability to said biomolecule. These compounds may also provide means for measurability and quantification of the labelled biomolecule.

In an embodiment, the label is attached to the biomolecule via a covalent bond. In a further embodiment, the biomolecule is an antibody, an antibody fragment (FAB), single chain variable fragment (scFC), a nanobody, an affibody, a minibody, a variable new antigen receptor (VNAR) single domain antibody fragment or small molecule for immunoassay. With direct detection, the label is attached via a covalent bond to the primary biomolecule. Alternatively, using indirect detection, the label is covalently attached to a secondary biomolecule, which is allowed to bind to the primary biomolecule during the immunoassay.

The interaction of streptavidin with biotin can be exploited for assay design, detection and diagnostic targeting. The extremely strong interaction between the biotin and streptavidin facilitates the selective labelling of streptavidin derivatised biomolecules with biotin-based labels, tags and probes. If the resultant streptavidin-biotin complex contains detection components then the targeted complex can be located and quantified. Biotinylation reagents can be derivatised with labels then covalently attached to targeting biomolecules such as antibodies.

In an embodiment, the label is a biotinylation reagent. In an embodiment, the label is a carboxylate-biotinylation moiety. In an embodiment, the label is a NHS-biotinylation moiety. In an embodiment, the label is NHS-biotin. In an embodiment, the label is Sulpho-NHS-biotin. In an embodiment, the label is NHS-LC-biotin. In an embodiment, the label is Sulpho-NHS-LC-biotin moiety.

In an embodiment, the label is a D-biotin reagent. In an embodiment, the label is a NHS-D-biotin moiety. In an embodiment, the label is a carboxylate-D-biotin moiety. In an embodiment, the label is a biocytin reagent. In an embodiment, the label is a NHS-biocytin moiety. In an embodiment, the label is a carboxylate-biocytin moiety.

In an embodiment, the label is NHS-iminobiotin. In an embodiment, the label is Sulpho-NHS-SS-biotin.

In an embodiment, the label is biotin-BMCC. In an embodiment, the label is biotin-HPDP. In an embodiment, the label is iodoacetyl-LC-biotin.

In an embodiment, the label is biotin-hydrazide. In an embodiment, the label is biotin-LC-hydrazide. In an embodiment, the label is biocytin hydrazide. In an embodiment, the label is 5-(biotinamido)pentylamine.

In an embodiment, the label is photobiotin. In an embodiment, the label is psoralen-PEO3-biotin.

In an embodiment, the label is p-aminobenzoyl biocytin.

In an embodiment, the label is a glycan biotinylation moiety. In an embodiment, the label is biotinylated diaminopyridine (BAP). In an embodiment, the label is biotinyl-L-3-(2-naphthyl)-alanine hydrazide. In an embodiment, the label is biotin-PEG-phosphine.

In an embodiment, the label is selected from the group consisting of: a complexing agent, a complexing agent bound to chelated metals and fluorophores. The label may be fluorescent compounds or compounds which may be detected by NMR or ESR spectroscopy.

The label may be a bifunctional chelating agent (BCA) capable of chelating metal ions in a coordination complex. The label may be a bifunctional chelating agent (BCA) capable of chelating radioactive metals in a coordination complex. In an embodiment, the label is a DTPA ligand capable of chelating radioactive metals in a coordination complex. In an embodiment, the label is a DOTA ligand capable of chelating radioactive metals in a coordination complex. In an embodiment, the label is a NOTA ligand capable of chelating radioactive metals in a coordination complex. In an embodiment, the label is a TETA ligand capable of chelating radioactive metals in a coordination complex. In an embodiment, the label is a DTTA ligand capable of chelating radioactive metals in a coordination complex. In an embodiment, the label is a DFA ligand capable of chelating radioactive metals in a coordination complex.

In an embodiment, the label is a chelated metal. Chelated metals include chelates of di- or tripositive metals having a coordination number from 2 up to 8 inclusive. Particular examples of such metals include technetium (Tc), rhenium (Re), cobalt (Co), copper (Cu), gold (Au), silver (Ag), lead (Pb), bismuth (Bi), indium (In), gallium (Ga), yttrium (Y), terbium (Tb), gadolinium (Gd), and Scandium (Sc). Preferably the metal is a radionuclide.

The chelated metal may be, for example, one of the above types of metal chelated with any suitable polydentate chelating agent, for example acyclic or cyclic polyamines, polyethers, (e.g. crown ethers and derivatives thereof); polyamides; porphyrins; and carbocyclic derivatives.

In general, the type of chelating agent will depend on the metal in use. One particularly useful group of chelating agents in conjugates according to the invention, however, are acyclic and cyclic polyamines, especially polyaminocarboxylic acids, for example diethylenetriaminepentaacetic acid and derivatives thereof, and macrocyclic amines, e.g. cyclic tri-aza and tetra-aza derivatives; and polyamides, especially desferrixoamine and derivatives thereof.

In an embodiment, the label is a radionuclide. Biomolecule-radionuclide-conjugates; specifically antibody-radionuclide-conjugates, have been used to image a variety of different cancers types. These conjugates have also been used to image tumour volume. Images of the tumour are obtained by using one or more combinations of the following detection methods: planar imaging; single photon emission computed tomography (SPECT) and positron emission tomography (PET). These techniques can be extended to hybrid imaging-using systems incorporating PET (or SPECT) with computed tomography (CT). More recently, PET scanners have also been hybridized—with a magnetic resonance imaging (MRI) instruments—to obtain a PET-MRI machine, already available in the clinical setting. Imaging of the tumour site (cancer) is achieved using antibody-reporter conjugates in which the antibody is used to direct the reporter payload (the radionuclide imaging agent) directly to the tumour through specific binding to appropriate antigens. When the tumour site retains an appropriate level of the antibody-radionuclide-conjugate the location of tumourous mass can be detected through the emitted radiation of the radionuclide isotope.

The choice of radionuclide payload for such immunodetection techniques is based on half-life, nature of the radiation emitted and the metabolic pathways of the radionuclide in the body as the conjugate degrades. Isotopes of iodine and divalent metal ions are the most commonly used radionuclides. Typically radionuclide reporter moieties are gamma or positron radiation emitters.

Immunoreactions between antigen-antibody can be visualised using microscopy techniques. The antibody is typically derivatised with a label including by example: enzyme, fluorophore, colloidal gold or biotin. An enzyme label can be visualized in the light microscope by means of enzyme histochemical methods via chromogenic reactions. In contrast, fluorophore labels can be directly visualized in a fluorescent microscope. Electron-dense labels such as colloidal gold are visible in the electron microscope without further treatment. Biotin labels can be exploited in multiple techniques including light, fluorescence and electron microscopy in combination with ABC technique. Like biotin, some other haptens, such as digoxigenin (DIG) or dinitrophenol (DNP), can also be coupled to antibodies to assist visualisation.

Typically labels are chemically introduced to biomolecules such as antibodies upon a variety of functional groups resent on the biomolecule. These functional groups are specifically targeted using appropriately activated label compounds. The biomolecule is labelled by chemical means via covalent attachment of the label via the targeted functional group on the biomolecule.

In undertaking fluorescent labelling of biomolecules researchers are able to detect specific components of complex biological assemblies with exquisite sensitivity in applications, including but not limited by: imaging, flow cytometry, Western blot assays, immunochemistry, fluorescence in situ hybridization (FISH), cell tracing, receptor labelling, etc. Fluorescent labels can also be used to probe biological structure, function and interactions of biomolecules. Fluorescent labelling of biomolecules is advantageous over other labelling techniques as the resultant conjugates can be detected at low concentration and the labelling procedures are non-destructive to the function and folding of the biomolecule. An expert review of classes of fluorophores and chemistry for introducing the label can be found in the following document (The Molecular Probes Handbook—‘A Guide to Fluorescent Probes & Labelling Technologies’, 2010, 11th Edition).

Fluorophores can be divided into three general groups: organic dyes, biological fluorophores and quantum dots.

Synthetic organic dyes, such as fluorescein, were the first fluorescent compounds used in biological research. Derivatives of these original compounds have been produced to improve their photostability and solubility. Due to the inherent small molecular weight of these dyes they offer distinct advantages over biological fluorophores. The small size of the dyes facilitates crosslinking with macromolecules, such as antibodies as the dye is small enough not to interfere with proper biological function.

Bioluminescence has been known for millennia but it wasn't until the 1990's that the first application of a biological fluorophore (green fluorescent protein) was investigated as a gene expression reporter. Since that time, derivatives of the green fluorescent protein, phycobiliproteins (allophycocyanin, phycocyanin, phycoerythrin and phycoerythrocyanin) and many other proteins have been designed for use in biological expression systems. Biological fluorophores are now commonplace in biological research.

Quantum dots are nanocrystals with unique chemical properties that provide tight control over the spectral characteristics of the fluorophore. Since development in the 1990's Quantum dots have been increasingly used for fluorescence applications in biological research. Quantum dots are nanoscale-sized (2-50 nm) semiconductors that, when excited, emit fluorescence at a wavelength based on the size of the particle. Smaller quantum dots emit higher energy than large quantum dots and subsequently the emitted light shifts from blue to red as the size of the nanocrystal increases. The size of the quantum dot can be tightly controlled and thus greater specificity for distinct excitation and emission wavelengths can be achieved when compared to other classes of fluorophore. Quantum dots have also been reported to be more photostable than other fluorophores. Furthermore, quantum dots can be coated for use in different biological applications such as protein labelling.

The benefit of these types of fluorophores is that expression plasmids can be introduced into either bacteria, cells, organs or whole organisms, to drive expression of that fluorophore either alone or fused to a protein of interest in the context of the biological processes studied. The use of fluorescent proteins can be time consuming, and expressing large amounts of light-producing proteins can cause reactive oxygen species and induce artifactual responses or toxicity. Additionally, the size of the fluorescent protein can change the normal biological function of the cellular protein to which the fluorophore is fused, and biological fluorophores do not typically provide the level of photostability and sensitivity offered by synthetic fluorescent dyes.

Fluorescent labels (Fluorophores) can be introduced to biomolecules by a variety of different chemistries. Typically Fluorophores may be activated and reactive to classes of corresponding functional groups on the biomolecule characterised as: amine reactive, thiol reactive. Furthermore, fluorophore labels may be introduced to biomolecules through functional group modification. These groups of fluorophores may be characterised as: click chemistry reactive, alcohol modification reagents, ketone & aldehyde modification reagents and carboxylic acid & carboxamides derivatisation reagents. Fluorophores may be introduced to biomolecules through biotin and other activated biotinylation reagents. These biotin-based fluorophores can be characterised as: amine reactive, amine reactive chromophoric biotin derivatives, thiol reactive. Furthermore, biotin-based fluorophore labels may be introduced to biomolecules through functional group modification. These groups of biotin-based fluorophores may be characterised as: click chemistry biotin alkynes and biotin azide reagents, histidine, serine and threonine modification reagents with biotin, aldehyde modification reagents with biotin, hydrazide based biotin reagents, hydroxylamine based biotin reagents, carboxylic acids modification reagents with biotin, Reactive DSB-X™ Biotin Derivatives.

Fluorophore labels may be introduced to biomolecules through the use of heterobifunctional or homobifunctional crosslinker derivatives. Crosslinkers are discreet chemical reagents defined for the purpose of creating intramolecular covalent bonds between one or more biomolecule species. Crosslinking reagents themselves are characterized by having at least two reactive functional groups as part of the compositional structure. For heterobifunctional crosslinkers these reactive functional groups are orthogonal to each other facilitating different reactivity of the terminal ends. These reactive ends are specifically targeted by component functional groups that exist on the biomolecule and/or the label. Focusing on the target reactive groups of the biomolecule a crosslinker can be divided into several characteristic modes of use: amine reactive, sulfhydryl reactive, carbohydrate reactive, and carboxyl reactive. Once one end of the crosslinker is covalently bound to the biomolecule the adjacent activated terminal end can then react with a label such as a fluorophore. Alternatively the heterobifunctional crosslinker can react with the label first, before being introduced to the biomolecule in the second step, for formation of the conjugate. Typically, the most labile reactive terminal of the crosslinker is conjugated to first. Examples of the utility of crosslinkers is expertly reviewed in the following documents (see ‘Bioconjugate Techniques’, Part III, Pages 745-1003, Greg T. Hermanson, 2008, Academic Press, ISBN: 978-0-12-370501-3, ‘Cross-linking Reagents—Technical Handbook’, Pierce/Thermo, www.piercenet.com, Protein Methods Library).

In contrast to chemical crosslinking reagents which are often used to prepare bioconjugates, photoreactive crosslinking reagents are important tools for determining the proximity of two sites. These probes can be employed to define relationships between two reactive groups that are on a single protein, on a ligand and its receptor or on separate biomolecules within an assembly. In the latter case, photoreactive crosslinking reagents can potentially reveal interactions among proteins, nucleic acids and membranes in live cells.

Photoreactive crosslinking reagents are a special subset of the heterobifunctional crosslinking reagents. Upon UV illumination, these reagents react with nucleophiles or form C—H insertion products. Photoreactive crosslinking reagents may be used to label biomolecules with fluorophores. Generally, photoactivatable crosslinkers are defined in two classes. Those that can be used to generate short-lived, high-energy intermediates that can chemically couple to nearby residues and ‘caged probes’ that are designed to be biologically inactive until UV light-mediated photolysis releases a natural product. Photolysis of each of these photoactivatable probes can be accomplished with high spatial and temporal resolution, releasing active probe at the site of interest.

Antibodies modified with fluorophore labels are valuable tools in immunology research. Antibody-fluorophore conjugates (AFC) present a non-toxic model of an antibody-drug conjugate (ADC). These conjugates are valuable molecular tools for mechanistic studies and PK evaluation as recently illustrated in several reports (Bruker, Application Note LCMS-94, Ben-Quan Shen et al, Nature Biotechnology, 2012, 30, 184; P. Strop et al, Chem. Biol. 2013, 20, 161; E. Wagner-Rousset et al, MAbs, 2014, 6, 1, 173).

In an embodiment, the label is an enzyme. In an embodiment, the label is colloidal gold. In an embodiment, the label is a biotin compound. In an embodiment, the label is a hapten. In an embodiment, the label is digoxigenin. In an embodiment, the label is dinitrophenol.

In an embodiment, the label is a fluorophore. In an embodiment, the label is an amine reactive fluorophore. In an embodiment, the label is a thiol reactive fluorophore. In an embodiment, the label is a click chemistry reactive fluorophore. In an embodiment, the label is a fluorophore-based alcohol modification reagent. In an embodiment, the label is a fluorophore-based ketone modification reagent. In an embodiment, the label is a fluorophore-based aldehyde modification reagent. In an embodiment, the label is a fluorophore-based carboxylic acid modification reagent. In an embodiment, the label is a fluorophore-based carboxamide modification reagent. In an embodiment, the label is an amine reactive biotin-based fluorophore. In an embodiment, the label is a thiol reactive biotin-based fluorophore. In an embodiment, the label is an amine reactive chromophoric biotin-based fluorophore. In an embodiment, the label is a click chemistry biotin-based fluorophore. In an embodiment, the label is a click chemistry alkyne biotin-based fluorophore. In an embodiment, the label is a click chemistry azide biotin-based fluorophore. In an embodiment, the label is a biotin-fluorophore-based histidine modification reagent. In an embodiment, the label is a biotin-fluorophore-based serine modification reagent. In an embodiment, the label is a biotin-fluorophore-based threonine modification reagent. In an embodiment, the label is a biotin-fluorophore-based aldehyde modification reagent. In an embodiment, the label is a biotin-fluorophore-based hydrazide modification reagent. In an embodiment, the label is a biotin-fluorophore-based hydroxylamine modification reagent. In an embodiment, the label is a biotin-fluorophore-based carboxylic acid modification reagent. In an embodiment, the label is a biotin-fluorophore-based reactive DSB-X™ modification reagent.

In an embodiment, the label is a fluorophore-based crosslinker. In an embodiment, the label is a fluorophore-based heterobifunctional crosslinker. In an embodiment, the label is a fluorophore-based homobifunctional crosslinker. In an embodiment, the label is a fluorophore-based photoreactive crosslinker. In an embodiment, the label is a fluorophore-based photoreactive heterobifunctional crosslinker. In an embodiment, the label is a fluorophore-based photoreactive homobifunctional crosslinker.

In an embodiment, the fluorophore is an organic dye. In an embodiment, the fluorophore is a xanthene dye. In an embodiment, the fluorophore is fluorescein. In an embodiment, the fluorophore is fluorescein isothiocyanate (FITC). In an embodiment, the fluorophore is NHS-fluorescein. In an embodiment, the fluorophore is sulphonated NHS-fluorescein. In an embodiment, the fluorophore is NHS-LC-fluorescein. In an embodiment, the fluorophore is sulphonated NHS-LC-fluorescein. In an embodiment, the fluorophore is 5-iodoacetamido-fluorescein. In an embodiment, the fluorophore is 6-iodoacetamido-fluorescein. In an embodiment, the fluorophore is fluorescein-5-maleimide. In an embodiment, the fluorophore is SAMSA-fluorescein. In an embodiment, the fluorophore is fluorescein-5-thiosemicarbazide. In an embodiment, the fluorophore is 5-(((2-(carbohydrazino)methyl)thio)acetyl)-aminofluorescein.

In an embodiment, the fluorophore is a coumarin. In an embodiment, the fluorophore is a benzopyrone. In an embodiment, the fluorophore is aminomethylcoumarin acetate (AMCA). In an embodiment, the fluorophore is 7-amino-4-methylcoumarin acetate (AMCA). In an embodiment, the fluorophore is NHS-AMCA. In an embodiment, the fluorophore is Sulfo-NHS-AMCA. In an embodiment, the fluorophore is AMCA-HPDP. In an embodiment, the fluorophore is DCIA. In an embodiment, the fluorophore is AMCA-hydrazide.

In an embodiment, the fluorophore is a rhodamine dye. In an embodiment, the fluorophore is a rhodamine B dye. In an embodiment, the fluorophore is a rhodamine 6G dye. In an embodiment, the fluorophore is a rhodamine 110 dye. In an embodiment, the fluorophore is a sulphorhodamine B dye. In an embodiment, the fluorophore is a sulphorhodamine 101 dye. In an embodiment, the fluorophore is tetramethylrhodamine (TRITC). In an embodiment, the fluorophore is tetramethylrhodamine isothiocyanate. In an embodiment, the fluorophore is NHS-rhodamine. In an embodiment, the fluorophore is lissamine rhodamine B sulphonyl chloride. In an embodiment, the fluorophore is tetramethylrhodamine-5-iodoacetamide. In an embodiment, the fluorophore is tetramethylrhodamine-6-iodoacetamide. In an embodiment, the fluorophore is lissamine rhodamine B sulphonyl hydrazine.

In an embodiment the fluorophore is Texas Red. In an embodiment the fluorophore is Texas Red Sulfonyl Chloride. In an embodiment the fluorophore is Texas Red hydrazine. In an embodiment the fluorophore is Oregon Green®. In an embodiment the fluorophore is Pacific Blue™. In an embodiment the fluorophore is Pacific Green™. In an embodiment the fluorophore is Pacific Orange™.

In an embodiment the fluorophore is a lanthanide chelate. In an embodiment the fluorophore is BCPDA. In an embodiment the fluorophore is TBP. In an embodiment the fluorophore is TMT. In an embodiment the fluorophore is BHHCT. In an embodiment the fluorophore is BCOT.

In an embodiment the fluorophore is cyanine dye. In an embodiment the fluorophore is a polymethine. In an embodiment the fluorophore is tricyanine dye. In an embodiment the fluorophore is pentacyanine dye. In an embodiment the fluorophore is heptacyanine dye. In an embodiment the fluorophore is sulphonated cyanine dye. In an embodiment the fluorophore is an indolium-based cyanine dye. In an embodiment the fluorophore is benzoindolium-based cyanine dye. In an embodiment the fluorophore is a pyridinium-based cyanine dye. In an embodiment the fluorophore is thiazolium-based cyanine dye. In an embodiment the fluorophore is quinolinium-based cyanine dye. In an embodiment the fluorophore is imidazolium-based cyanine dye. In an embodiment the fluorophore is a NHS-ester cyanine dye. In an embodiment the fluorophore is a maleimide cyanine dye. In an embodiment the fluorophore is a hydrazide cyanine dye.

In an embodiment the fluorophore is CyDye. In an embodiment the fluorophore is Alexa Fluor. In an embodiment the fluorophore is LI-COR. In an embodiment the fluorophore is Alexa Fluor 488. In an embodiment the fluorophore is Atto dye. In an embodiment the fluorophore is FAM™. In an embodiment the fluorophore is JOE™. In an embodiment the fluorophore is TET™. In an embodiment the fluorophore is Alexa Fluor 532. In an embodiment the fluorophore is HEX™. In an embodiment the fluorophore is TAMRA™. In an embodiment the fluorophore is Cy3. In an embodiment the fluorophore is Cy3.5. In an embodiment the fluorophore is ROX™. In an embodiment the fluorophore is Alexa Fluor 594. In an embodiment the fluorophore is Alexa Fluor 633. In an embodiment the fluorophore is Cy5. In an embodiment the fluorophore is Alexa Fluor 647. In an embodiment the fluorophore is Cy5.5

In an embodiment the fluorophore is a cascade blue dye. In an embodiment the fluorophore is a cascade blue acetyl azide. In an embodiment the fluorophore is a cascade blue cadaverine. In an embodiment the fluorophore is a cascade blue ethylenediamine. In an embodiment the fluorophore is a cascade blue hydrazide.

In an embodiment the fluorophore is a lucifer yellow dye. In an embodiment the fluorophore is a lucifer yellow iodoacetamide. In an embodiment the fluorophore is a lucifer yellow CH.

In an embodiment the fluorophore is a Dylight dye. In an embodiment the fluorophore is a Dylight 350. In an embodiment the fluorophore is a Dylight 404. In an embodiment the fluorophore is a Dylight 488. In an embodiment the fluorophore is a Dylight 550. In an embodiment the fluorophore is a Dylight 595. In an embodiment the fluorophore is a Dylight 633. In an embodiment the fluorophore is a Dylight 650. In an embodiment the fluorophore is a Dylight 680. In an embodiment the fluorophore is a Dylight 755. In an embodiment the fluorophore is a Dylight 800.

In an embodiment the fluorophore is an Atto dye. In an embodiment the fluorophore is Atto 390. In an embodiment the fluorophore is Atto 425. In an embodiment the fluorophore is Atto 465. In an embodiment the fluorophore is Atto 488. In an embodiment the fluorophore is Atto 520. In an embodiment the fluorophore is Atto 532. In an embodiment the fluorophore is Atto 550. In an embodiment the fluorophore is Atto 565. In an embodiment the fluorophore is Atto 590. In an embodiment the fluorophore is Atto 594. In an embodiment the fluorophore is Atto 610. In an embodiment the fluorophore is Atto 611X. In an embodiment the fluorophore is Atto 620. In an embodiment the fluorophore is Atto 633. In an embodiment the fluorophore is Atto 647. In an embodiment the fluorophore is Atto 647N. In an embodiment the fluorophore is Atto 655. In an embodiment the fluorophore is Atto 665. In an embodiment the fluorophore is Atto 700. In an embodiment the fluorophore is Atto 725. In an embodiment the fluorophore is Atto 680. In an embodiment the fluorophore is Atto Oxa12. In an embodiment the fluorophore is Atto Rho101. In an embodiment the fluorophore is Atto Rho6G. In an embodiment the fluorophore is Atto Rho11. In an embodiment the fluorophore is Atto Rho12. In an embodiment the fluorophore is Atto Rho13. In an embodiment the fluorophore is Atto Rho14.

In an embodiment the fluorophore is a dipyrromethene complexed with a disubstituted boron atom. In an embodiment the fluorophore is boron-dipyrromethene dye. In an embodiment the fluorophore is BODIPY dye. In an embodiment the fluorophore is BODIPY FL C3-SE. In an embodiment the fluorophore is BODIPY 530/550 C3. In an embodiment the fluorophore is BODIPY 530/550 C3-SE. In an embodiment the fluorophore is BODIPY 530/550 C3 Hydrazide. In an embodiment the fluorophore is BODIPY 593/503 C3 Hydrazide. In an embodiment the fluorophore is BODIPY FL C3 Hydrazide. In an embodiment the fluorophore is BODIPY FL IA. In an embodiment the fluorophore is BODIPY 530/550 IA. In an embodiment the fluorophore is bromo-BODIPY 494/503.

SETA, SeTau & Square are a series of dicyanomethylene squaraine-based dyes that span the whole visible spectrum and the near infra-red. In an embodiment the fluorophore is Seta-375. In an embodiment the fluorophore is Seta-470. In an embodiment the fluorophore is Seta-555. In an embodiment the fluorophore is Seta-632. In an embodiment the fluorophore is Seta-633. In an embodiment the fluorophore is Seta-635. In an embodiment the fluorophore is Seta-646. In an embodiment the fluorophore is Seta-660. In an embodiment the fluorophore is Seta-670. In an embodiment the fluorophore is Seta-680. In an embodiment the fluorophore is Seta-700. In an embodiment the fluorophore is Seta-750. In an embodiment the fluorophore is Setau-405.

In an embodiment the fluorophore is Lightning-Link®. In an embodiment the fluorophore is Lightning-Link® Rapid.

In an embodiment the fluorophore is a quantum dot. In an embodiment the fluorophore is a Qdot® probe. In an embodiment the fluorophore is Qdot® 525. In an embodiment the fluorophore is Qdot® 565. In an embodiment the fluorophore is Qdot® 605.

In an embodiment the fluorophore is Qdot® 655. In an embodiment the fluorophore is Qdot® 705. In an embodiment the fluorophore is Qdot® 800.

In an embodiment the fluorophore is a biological fluorophore. In an embodiment the fluorophore is an enzyme-based biological fluorophore. In an embodiment the fluorophore is a protein-based biological fluorophore. In an embodiment the fluorophore is an expressed fluorescent protein. In an embodiment the fluorophore is Cyan Fluorescent Protein (CFP). In an embodiment the fluorophore is Green Fluorescent Protein (GFP). In an embodiment the fluorophore is Red Fluorescent Protein (RFP). In an embodiment the fluorophore is a phycobiliprotein. In an embodiment the fluorophore is R-Phycoerythrin Fluorescent Protein. In an embodiment the fluorophore is Allophycocyanin (APC). In an embodiment the fluorophore is phycocyanin. In an embodiment the fluorophore is phycoerythrin. In an embodiment the fluorophore is a phycobiliprotein tandem dye.

In an embodiment the reporter group is a tag. The term ‘tag’ can be used synonymously with the term ‘label’ herein.

Effector Group

The term ‘effector group’ in the conjugates according to the present invention includes any group or compound which is capable of eliciting a change in, or a response from, a biological system and which also confers this property to the conjugates of the invention. An effector compound may confer properties to the conjugate not naturally occurring in the biomolecule.

In an embodiment, the term ‘effector group’ excludes any substance that, when administered into the body of a living organism, alters normal bodily function. In an embodiment, the term ‘effector group’ excludes a substance used in the treatment, cure, prevention, or diagnosis of a disease and/or a substance used to otherwise enhance physical or mental well-being. In a particular embodiment, the term ‘effector group’ excludes a cytotoxic drug.

In an alternative embodiment, effector groups may be any group or compound that elicits a change in or response from a biological system to a biomolecule-effector-conjugate. In an embodiment the biomolecule conjugate is an antibody-effector-conjugate, modified antibody-effector-conjugate or antibody fragment-effector-conjugate.

Suitable effector molecules include antibiotics, anti-inflammatory, non-steriodal anti-inflammatory drug (NSAID), cytokines, radionuclides, enzymes, antibody directed enzyme prodrug therapy (ADEPT), Boron-10 (for Boron Neutron Capture Therapy), Gadolinium-157 (for Gadolinium Neutron Capture Therapy), proteins, peptides, polypeptides, modified peptides, peptide nucleic acids (PNAs), siRNA, viruses, oligonucleotides, modified oligonucleotides, oligonucleotide-drug conjugates, nucleotides, nucleosides, purines, pyrimidines, liposomes, nanocarriers, complexing agent binding a metallic, particulates, metalloproteins, peptide-drug conjugates, peptide-oligonucleotide hybrids, amino acids, non-naturally occurring amino acids, diamino acids, synthetic amino acids, amino acid-drug conjugates, oligosaccharides, polysaccharide, disaccharides, monosaccharides, amino sugars, lipids, phospholipids, glycolipids, sterols, vitamins, hormones, steroids, neurotransmitters, carbohydrates, sugars, viruses, cells, killer cells, active pharmaceutical ingredients (APIs), and precursor compounds or a derivatives of any of these.

Since its inception in the early 1980's antibody directed enzyme prodrug therapy (ADEPT) provides a technique to selectively deliver effector moieties such as chemotherapeutic payloads to cancer sites. The basic principle of ADEPT is differential to that of antibody-drug-conjugates (ADCs). The ADEPT technique covalently attaches an enzyme to an antibody directed to a tumour associated antigen. Once administered, the enzyme-antibody conjugate accumulates and binds to the tumour cells to which the antibody has affinity. Once docked at the antigen superfluous antibody-enzyme-conjugate is cleared from the blood and normal tissues. Once clearance is complete a non-toxic prodrug is given to which the enzyme has specificity. The targeted enzyme converts the non-toxic prodrug into a potent cell killing agent within tumours to achieve effective therapy without normal tissue toxicity. A key feature of ADEPT is the amplification effect as one molecule of enzyme catalyses the conversion of many molecules of the prodrug to the potent cell killing agent. ADEPT enables higher drug concentrations at the tumour compared with the direct injection of the drug alone. The ADEPT technique can also participate in the bystander effect delivering potent cell killing agents in a localised area to the tumour to cells not expressing the characteristic tumour antigen (I. Niculescu-Duvaz et al, Advanced Drug Delivery Reviews, 1997, 26, 151).

ADEPT is differential from the ADC technique in that the ADC technique forms a covalent bond with an antibody and potent drug moiety. ADEPT requires the conversion of the prodrug to a potent active via the antibody-enzyme conjugate accumulated at the tumour. The prodrug is administered in-vivo and does not form part of the conjugate composition in the ADEPT technique.

In an embodiment, the effector is an enzyme. In an embodiment, the effector is an antibody-enzyme conjugate. In a further embodiment, the effector is an antibody-enzyme conjugate for ADEPT.

The past decade has seen a marked trend in bacterial resistance to traditional antibiotics. This has necessitated a concerted effort to develop new antimicrobial agents, preferentially with novel modes of action and/or different cellular targets compared with the existing antibiotics. As a result, new classes of compounds designed to avoid defined resistance mechanisms are undergoing pre-clinical and clinical evaluation. A recent approach is the use of antibody-antibiotic-conjugates (AACs) for the treatment of infectious diseases. U.S. Pat. No. 5,545,721 reports the use of AACs for the prevention and treatment of blood-borne and toxin mediated diseases, in particular the prevention and treatment of sepsis. US2002168368 presents a novel approach for treating bacterial infection and disease, particularly streptococcal infection resulting from Staphylococcus aureus and most significantly methicillin-resistant S. aureus and vancomycin-resistant S. aureus infections. The AACs presented therein describes compositions of enriched antibodies in which the antibody component contains both an antigen-binding portion specific for a bacterial antigen (e.g., a S. aureus antigen) and a constant region that does not bind a bacterial Fc-binding protein. Preparations of such antibodies should be effective for treating infections caused by bacteria that express Fc-binding proteins.

In an embodiment, the effector is an antibiotic. In an embodiment, the effector is moiety which exhibits a bactericidal or bacteriostatic effect. In an embodiment, the effector is moiety which exhibits an anti-parasitic effect.

In an embodiment, the effector is an antibody-antibiotic-conjugate. In a further embodiment, the effector is an antibody-antibiotic-conjugate for treating infection. In a further embodiment, the effector is an antibody-antibiotic-conjugate for treating sepsis. In a further embodiment, the effector is an antibody-antibiotic-conjugate for treating infection resulting from Staphylococcus aureus.

In an embodiment, the effector is chloramphenicol. In an embodiment, the effector is erythromycin. In an embodiment, the effector is lincomycin. In an embodiment, the effector is fusidic acid. In an embodiment, the effector is streptomycin. In an embodiment, the effector is aminoglycoside antibiotic. In an embodiment, the effector is tetracycline. In an embodiment, the effector is polymyxin. In an embodiment, the effector is fosfomycin. In an embodiment, the effector is vancomycin. In an embodiment, the effector is ristocetin. In an embodiment, the effector is bacitracin. In an embodiment, the effector is gramicidin. In an embodiment, the effector is penicillin. In an embodiment, the effector is cephalosporin. In an embodiment, the effector is macrolide. In an embodiment, the effector is ketolide. In an embodiment, the effector is sulphonamide. In an embodiment, the effector is trimethoprim. In an embodiment, the effector is quinolone. In an embodiment, the effector is lipopeptide. In an embodiment, the effector is glycopeptide. In an embodiment, the effector is fluoroquinolone. In an embodiment, the effector is lipoglycopeptide. In an embodiment, the effector is macrocyclic. In an embodiment, the effector is monobactam. In an embodiment, the effector is carbapenem. In an embodiment, the effector is beta-lactam. In an embodiment, the effector is lincosamide. In an embodiment, the effector is streptogramin. In an embodiment, the effector is oxazolidinone. In an embodiment, the effector is rifamycin. In an embodiment, the effector is polypeptide. In an embodiment, the effector is tuberactinomycin. In an embodiment, the effector is nitrofuran.

RNA interference (RNAi) is a new strategy for the development of antisense therapeutics that knock down gene expression in biological systems post-transcriptionally. RNAs are the direct products of genes and two classes are central to RNAi interference: microRNA (miRNA) and small interfering RNA (siRNA). RNAi interference may proceed through a mechanism whereby siRNA may degrade the target RNA, or miRNA may instigate a mechanism causing translation arrest of the target RNA. RNA interference has an important role in defending cells against parasitic nucleotide sequences such as viruses and transposons. There are two types of RNAi-based therapeutics: DNA-based RNAi and RNA-based RNAi. For an expert review see (N. Dias et al, Mol. Cancer Ther., 2002; 1, 347-355).

Potential RNAi-based therapies suffer from an inherent weakness from translation from in-vitro models towards the goal of FDA approved moieties for clinical in-vivo use. Drug delivery of RNAi is a formidable challenge. The component nucleic acids in RNAi are highly negatively charged and do not cross biological cell membranes by free diffusion. Therefore, in order to overcome this issue any in vivo delivery of RNAi therapeutics must use a targeting technology that enables the RNAi therapeutic to traverse biological membrane barriers.

Various delivery approaches have been tested to overcome the issue of traversing cell membrane barriers to limited degrees of success. These have included liposomal formulations of siRNA with cationic lipids and conjugation of siRNA to cell-penetrating peptides. After crossing the cell membrane, siRNA is often trapped inside endosomes where they are unavailable for antisense purposes. Thus, by attaching siRNA to endosomolytic peptides the endosomal membranes may be destabilised allowing the siRNA payload to escape into the cytoplasm, where the siRNA can be incorporated into the RNA induced silencing complex. In addition, systemically delivered siRNA needs to avoid uptake and clearance by non-target tissues.

One approach to overcome this rate-limiting role is to use a conjugate system in which the RNAi payload is delivered via an antibody or a combination of antibody and biotin/streptavidin labelling. W. Pardridge (Advanced Drug Delivery Reviews, 2007, 59, 14) describes such an approach, which in turn is similar to that employed for delivery of antisense agents. In this review SiRNA duplexes were conjugated to targeting antibodies to overcome delivery of this nucleic therapeutic, firstly over the brain capillary endothelial wall; which forms the blood-brain barrier (BBB), and secondly then traverse the brain cell plasma membrane barriers.

M. Tan et al (Anal. Biochem. 2012, 430, 171) reports a similar approach using antibody-siRNA-conjugates in which siRNA was covalently conjugated to antibodies that bind to specific cell surface proteins and internalise. The study focused on understanding the function and behaviour of antibody-siRNA-conjugates in vivo through pharmacokinetic analysis.

siRNA is degraded in vivo by ribonucleases within a few hours and therefore a chemical conjugate modification extends plasma half-life and therefore efficacy. Antibody-siRNA-conjugates present excellent opportunities to overcome some of the inherent delivery and stability issues of siRNA therapies noted above.

The potential applications for antisense oligonucleotides such as RNAi are limited only by the genetic information available. RNAi can be developed against any target in which the inhibition of protein production or the inhibition of RNA processing yields the therapeutic result.

In an embodiment, the effector is a RNA moiety. In an embodiment, the effector is a RNA interfering agent (RNAi). In an embodiment, the effector is a DNA-based RNAi. In an embodiment, the effector is a RNA-based RNAi. In an embodiment, the effector is a siRNA. In an embodiment, the effector is a miRNA. In an embodiment, the effector is a shRNA.

In an embodiment, the effector is an antisense moiety. In an embodiment, the effector is an oligonucleotide. In an embodiment, the effector is a single-stranded short sequence of DNA bases. In an embodiment, the effector is a single-stranded short sequence of greater than 11 DNA bases. In an embodiment, the effector is an oligodeoxynucleotide. In an embodiment, the effector is a phosphorohioate oligonucleotide. In an embodiment, the effector is a phosphoramidate oligonucleotide. In an embodiment, the effector is a 2′-O-methyl and 2′-O-methoxyethyl RNA. In an embodiment, the effector is a 2′-O-alkyl substituted phosphorothioate oligonucleotide. In an embodiment, the effector is a Peptide Nucleic Acid oligonucleotide (PNA). In an embodiment, the effector is a Locked Nucleic Acid oligonucleotide (LNA). In an embodiment, the effector is a Bridged Nucleic Acid oligonucleotide (BNA). In an embodiment, the effector is a Hexitol Nucleic Acid oligonucleotide (HNA). In an embodiment, the effector is a Morpholino oligonucleotide. In an embodiment, the effector is a ribozyme. In an embodiment, the effector is a hammerhead ribozyme. In an embodiment, the effector is a hairpin ribozyme.

In an embodiment, the effector is a non-viral gene therapy.

Cytokines are a large family of >100 small immunostimulatory proteins, typically 5-20 kDa, that function as short-range mediators. They are a unique class of intercellular regulatory proteins are involved in essentially every important biological process, ranging from cell proliferation to inflammation, immunity, migration, fibrosis, repair, and angiogenesis. Cytokines include chemokines, interferons, interleukins, lymphokines, tumour necrosis factor but generally not hormones or growth factors. Cytokines have been found to be important rate-limiting signals, and it is now known that blocking some cytokines, e.g., TNF-α, and cytokine receptors, such as human EGFR 1 (HER1) or HER2, can influence effective therapeutics.

Due to their multiple functions, including regulatory and effector cellular function in many diseases, these molecules, their receptors, and their signal transduction pathways are promising candidates for therapeutic interference. The therapeutic administration of cytokines, modulation of cytokine action, or at times gene therapy is being used for a wide range of infectious and autoimmune diseases.

Several cytokines have been investigated in clinical trials, led primarily through potent therapeutic activity observed in animal models of cancer and other diseases. However, substantial off-site/systemic toxicities are often reported at low doses, thus preventing escalation to therapeutically active regimens. This is coupled with only modest efficacies as a balance between maximum tolerated dose and minimum effective dose is necessary to achieve an appropriate, yet narrow, therapeutic window. In recent years, the use of recombinant antibodies or antibody fragments as delivery vehicles to create antibody-protein conjugates (more specifically, anti body-cytokine-conjugates or immunocytokines) promises to enhance greatly the therapeutic index of pro-inflammatory and anti-inflammatory cytokines.

US2010297060 discloses methods and compositions for the preparation of immunocytokines for targeting the treatment of tumours in-vivo. Also disclosed is a method for enhancing the uptake of immunocytokines into tumours based on a commination therapy of the immunocytokine with an immunocytokine uptake enhancing agent

WO03092737 describes a pharmaceutical composition of an antibody-cytokine-conjugate for diagnosis or treatment of cancer. Due to the complexity of the multivalent interactions of the antibody for the targeting antigen and the cytokine for its target vascular receptor in this example, combinations of antibody-cytokines are described that improve the therapeutic index of the cytokine moiety.

T. Hemmerle et al (Journal of Dermatological Science, 2014, 76, 96-103) reports the use of an immunocytokine conjugate for the treatment of chronic inflammatory conditions. In this approach an anti-EDA antibody F8 was used to selectively deliver interleukin 4 to the neo-vasculature. The technique has the potential to be expanded to deliver further immunomodulatory cytokines to sites of inflammatory skin conditions in the acute and chronic phase.

Skin inflammatory lesions can be selectively targeted using anti-EDA antibody-cytokine fusion proteins and the pharmacodelivery of IL4 confers a therapeutic benefit by shifting the cytokine balance.

Interleukin-2 fusion proteins account for the most advanced immunocytokines in clinical development (see US2014219920). This is relative as the unconjugated cytokine is routinely used for the therapy of patients with metastatic renal cell carcinoma or melanoma. Other cytokines employed as immunocytokine conjugates in clinical development include Interleukin-12 (US2014170109), TNF alpha for various cancer treatments and Interleukin-10 for treatment of arthritis. For an expert review see (N. Pasche, Drug Discovery Today, Volume 17, 11/12, June 2012).

In an embodiment, the effector is a cytokine moiety.

In an embodiment, the effector is Interleukin-2. In an embodiment, the effector is Interleukin-4. In an embodiment, the effector is Interleukin-7. In an embodiment, the effector is Interleukin-9. In an embodiment, the effector is Interleukin-15. In an embodiment, the effector is Interleukin-21.

In an embodiment, the effector is an interferon.

In an embodiment, the effector is a Type I interferon. In an embodiment, the effector is a Type II interferon. In an embodiment, the effector is a Type III interferon.

In an embodiment, the effector is a class-2 cytokine. In an embodiment, the effector is Interleukin-10. In an embodiment, the effector is Interleukin-19. In an embodiment, the effector is Interleukin-20. In an embodiment, the effector is Interleukin-22. In an embodiment, the effector is Interleukin-24 (Mda-7). In an embodiment, the effector is Interleukin-26. In an embodiment, the effector is interferon-alpha. In an embodiment, the effector is interferon-beta. In an embodiment, the effector is interferon-epsilon. In an embodiment, the effector is interferon-kappa. In an embodiment, the effector is interferon-omega. In an embodiment, the effector is interferon-delta. In an embodiment, the effector is interferon-tau. In an embodiment, the effector is interferon-gamma. In an embodiment, the effector is limitin. In an embodiment, the effector is interleukin-28A. In an embodiment, the effector is interleukin-28B. In an embodiment, the effector is interleukin-29.

In an embodiment, the effector is a non-immunological cytokine. In an embodiment, the effector is erythropoietin (EPO). In an embodiment, the effector is thrombopoietin (TPO).

In an embodiment, the effector is interleukin-1. In an embodiment, the effector is interleukin-1 alpha. In an embodiment, the effector is interleukin-1 beta. In an embodiment, the effector is interleukin-1Ra. In an embodiment, the effector is interleukin-18. In an embodiment, the effector is interleukin-38Ra. In an embodiment, the effector is interleukin-36 alpha. In an embodiment, the effector is interleukin-37. In an embodiment, the effector is interleukin-36 beta. In an embodiment, the effector is interleukin-36 gamma. In an embodiment, the effector is interleukin-38. In an embodiment, the effector is interleukin-33.

In an embodiment, the effector is interleukin-17. In an embodiment, the effector is interleukin-17A. In an embodiment, the effector is interleukin-17B. In an embodiment, the effector is interleukin-17C. In an embodiment, the effector is interleukin-17D. In an embodiment, the effector is interleukin-17E. In an embodiment, the effector is interleukin-17F.

In an embodiment, the effector is TNF. In an embodiment, the effector is TNF alpha. In an embodiment, the effector is LT-alpha. In an embodiment, the effector is LT-beta. In an embodiment, the effector is T cell antigen gp39 (CD40L). In an embodiment, the effector is CD27L. In an embodiment, the effector is CD30L. In an embodiment, the effector is FASL. In an embodiment, the effector is 4-1BBL. In an embodiment, the effector is OX40L. In an embodiment, the effector is TNF-related apoptosis inducing ligand (TRAIL).

In an embodiment, the effector is CD40LG (TNFSF5). In an embodiment, the effector is CD70 (TNFSF7). In an embodiment, the effector is EDA. In an embodiment, the effector is FASLG (TNFSF6). In an embodiment, the effector is LTA (TNFSF1). In an embodiment, the effector is LTB (TNFSF3).

In an embodiment, the effector is TNFSF4 (OX40L). In an embodiment, the effector is TNFSF8 (CD153). In an embodiment, the effector is TNFSF9. In an embodiment, the effector is TNFSF10 (TRAIL). In an embodiment, the effector is TNFSF11 (RANKL). In an embodiment, the effector is TNFSF12 (TWEAK). In an embodiment, the effector is TNFSF13. In an embodiment, the effector is TNFSF13B. In an embodiment, the effector is TNFSF14. In an embodiment, the effector is TNFSF15. In an embodiment, the effector is TNFSF18.

In an embodiment, the effector is TGF-beta. In an embodiment, the effector is a member of the TGF-beta superfamily. In an embodiment, the effector is a member of the decapentaplegic-Vg-related (DVR) related subfamily. In an embodiment, the effector is a member of the activin/inhibin subfamily. In an embodiment, the effector is a member of the TGF-beta subfamily.

In an embodiment, the effector is a chemokine. In an embodiment, the effector is a lymphokine. In an embodiment, the effector is tumour necrosis factor.

Lyposomes are microscopic, man-made spherical vesicles composed of a lamellar phase lipid bilayer. They are used as sustained-action delivery vehicles for a wide variety of payloads, including: drugs, vaccines, enzymes, non-enzyme proteins, genetic materials and nutritional supplements. The molecular payload is encapsulated inside these vesicles which eventually break down through natural processes, introducing the payload content into the bloodstream or into tissues to which they have migrated by diffusion through the walls of capillaries.

Liposomes are useful drug delivery vehicles since they may protect encapsulated drugs from enzymatic degradation and rapid clearance in vivo, or alter bio-distribution, potentially leading to reduced toxicities. Similarly to other biomolecule-effector-conjugates and biomolecule-reporter-conjugates the major issue to the development of these moieties for many specialised applications is the directed targeting of the liposome to the desired site whereby otherwise they would not accumulate. Consequently, a great deal of effort has been made over the years to develop liposomes that have targeting vectors attached to the bilayer surface. Liposome conjugates with various targeting vectors have been developed and evaluated including: oligosaccharides, peptides, proteins, vitamins and antibodies. Antibodies are ideal targeting vectors as they can be engineered (and manufactured) to recognise specific antigens expressed on the cell of interest (S. Ansell et al, Methods in Molecular Medicine, 2000, Vol. 25, 51-67. Drug Targeting: Strategies, Principles, and Applications. Ed. G. Francis & C. Delgado. Humana Press Inc.).

Immunoliposomes are biomolecule-effector-conjugates generated by conjugating antibodies either; (i) directly to lipid bilayer of liposomes in presence or absence of PEG chains (type I immunoliposomes) or (ii) to the distal end of the PEG chain (type II immunoliposomes).

Conjugation of antibodies directly to the lipid bilayer of a PEG containing liposomes (type I immunoliposomes) can result in reduced or even diminished antigen bonding, depending on the amount of incorporated PEG and the length of the PEG chains. However, antigen binding properties of immunoliposomes can be restored by conjugating the antibody to the terminus of the PEG chain, and therefore most of the recently developed immunoliposomes are based on type II immunoliposomes.

Recognition of the targeted cell by immunoliposomes is influenced by two factors. Firstly, the type antibody moiety (e.g. whole antibody, FAB, scFv, etc.) and secondly, the chemistry of the conjugation step influencing the homogeneity of the conjugate (e.g. stochastic conjugation vs site specific conjugation).

It has been extensively shown that whole antibodies coupled to liposomes are highly immunogenic. Such liposomes are rapidly eliminated through Fc-mediated phagocytosis by macrophages of the liver and spleen, and also by tumour localized macrophages.

Stochastic conjugation methods, by example; using thiolated antibody coupled to maleimide PEG lipids or using modified amino reactive PEG lipids, risk antibody inactivation and liposome aggregation by crosslinking. The disadvantages of using whole antibody can be circumvented by the use of antibody fragments such as fragment antigen binding (Fab) or single chain fragment variable (scFv) moieties.

Fab fragments have an average molecular weight of approximately 50 kDa and expose one or several solvated, surface accessible thiol groups, depending on the method of production. For comparison, Fab type II immunoliposomes have reduced immunogenicity compared to whole antibody type II immunoliposomes. Furthermore, whole antibody (IgG) type II immunoliposomes are cleared faster than Fab type II immunoliposomes. Pastorino et al (Cancer Res., 2003, 63, 1, 86-92) reported that Fab type II immunoliposomes have approximately two fold reduced immunogenicity compared with whole IgG type II immunoliposomes. The rate of elimination was three fold faster for whole IgG type II immunoliposomes compared with Fab type II immunoliposomes.

scFv fragments are approximately 25 kDa and are the smallest fragments to contain the entire antigen binding site of an antibody. They are formed by connecting the variable heavy and light chain domains with a short peptide linker with 15-20 amino acids.

In order to conjugate scFv fragments to liposomes, one or more additional cysteine residues are attached to the C terminus of scFv fragments. This allows for site-directed conjugation with the reactive thiol groups located opposite the antigen binding sites. This elaborate technique facilitates a similar conjugation process to that described above for Fab fragments. Conjugation of scFv fragments does not interfere with target cell recognition.

In order to achieve efficient conjugation to the liposome payload the scFv fragment is prepared by mild reduction to remove all dimeric species prior to the conjugation event. Antibodies, modified-antibodies and antibody fragments, modified-antibody fragments are coupled to liposomes by covalent bonds.

In direct coupling chemistries the antibody or antibody fragment is introduced to a reactive pre-formed liposome moiety (e.g. maleimide-PEG-liposome). However, direct coupling can result in lower coupling efficiencies, especially for scFv fragments. Alternatively, a post insertion method can be used to prepare immunnoliposome conjugates. In this method the antibody or antibody fragment is firstly conjugated to maleimide-PEG micelles. This method reports higher coupling efficiencies and an improved maintenance of immunoreactivity of the antibody.

Drug targeting with immunoliposomes is highly complex and influenced by various parameters and both the target site and the liposome.

Akin to other antibody-effector-conjugates the therapeutic efficiency of immunoliposomes is influenced by antigen density upon the targeted cells. A high density of target antigens increases delivery of the effector payload to the cell. However, antigen density on target cells is often low.

Drug loaded type II immunoliposomes containing antibodies that target an internalized antigen such as CD19 showed much more potency compared to immunoliposomes containing antibodies that target a non internalized antigen such as CD20. This study shows that internalization of immunoliposomes is a prerequisite for the induction of efficient cytotoxicity.

The therapeutic efficacy of immunoliposomes is dependent on the rate of release of payload and the lipid composition of the liposomes. Unfortunately, the major problem with immunoliposomes is their poor extravasation properties. Extravasation is antibody independent and it is the rate limiting step in the tumour cell targeting of solid tumors. This restricts the applications of immunoliposomes to those cancers in which tumour cells are readily accessible such as hematological malignancies or minimal residual diseases (liquid tumours).

New approaches have been developed to mitigate the poor extravasation of immunoliposomes by combining immunoliposomes with vascular targeting. In this approach liposomes are targeted to cells associated with neovascularization in tumour tissue as all solid tumours are reliant on neovascularization to grow. Furthermore, tumour blood vessels are easily accessible for immunoliposomes. Endothelial cells are genetically stable and should not become resistant to immunoliposomes therapy. Several antibodies and antibody fragments recognize antigens associated with endothelial cell activation and proliferation. These antigens include E-selectin, vascular endothelial growth factor receptor-2, the fibronectin splice variant ED-B, endoglin (CD105), vascular cell adhesion molecule-1 and tumor endothelial marker 1. In vivo study of anti-ED-B scFv immunoliposomes showed 62-90% reduction of tumour growth in F9 teratocarcinoma bearing mice in comparison to animals treated with untargeted control liposomes.

In an embodiment, the effector is a phospholipid-based vesicle. In an embodiment, the effector is a liposome. In an embodiment, the effector is a small unilamellar vesicle (SUV) liposome. In an embodiment, the effector is a large unilamellar vesicle (LUV) liposome. In an embodiment, the effector is a giant unilamellar vesicle (GUV) liposome. In an embodiment, the effector is a large multilamellar vesicle (MLV) liposome. In an embodiment, the effector is a multivesicular vesicle (MVV) liposome.

In a further embodiment, the effector is a liposome containing a second effector payload. In a further embodiment, the effector is a liposome containing a second drug-based effector payload. In a further embodiment, the effector is a liposome containing a second vaccine-based effector payload. In a further embodiment, the effector is a liposome containing a second enzyme-based effector payload. In a further embodiment, the effector is a liposome containing a second peptide-based effector payload. In a further embodiment, the effector is a liposome containing a second non-enzyme protein-based effector payload. In a further embodiment, the effector is a liposome containing a second oligonucleotide-based effector payload. In a further embodiment, the effector is a liposome containing a second RNAi-based effector payload. In a further embodiment, the effector is a liposome containing a second nutritional supplement-based effector payload. In a further embodiment, the effector is a liposome containing a second vitamin-based effector payload. In a further embodiment, the effector is a liposome containing a second active pharmaceutical ingredient (API)-based effector payload. In a further embodiment, the effector is a liposome containing a second small molecule-based effector payload. In a further embodiment, the effector is a liposome containing a second antibiotic-based effector payload. In a further embodiment, the effector is a liposome containing a second anti-inflammatory-based effector payload. In a further embodiment, the effector is a liposome containing a second-based effector payload. In a further embodiment, the effector is a liposome containing a second non-steriodal anti-inflammatory drug (NSAID)-based effector payload. In a further embodiment, the effector is a liposome containing a second cytokine-based effector payload. In a further embodiment, the effector is a liposome containing a second radionuclide-based effector payload. In a further embodiment, the effector is a liposome containing a second boron-10-based effector payload. In a further embodiment, the effector is a liposome containing a second gadolinium-157-based effector payload. In a further embodiment, the effector is a liposome containing a second peptide nucleic acid (PNA)-based effector payload. In a further embodiment, the effector is a liposome containing a second nucleotide-based effector payload. In a further embodiment, the effector is a liposome containing a second nucleoside-based effector payload. In a further embodiment, the effector is a liposome containing a second purine-based effector payload. In a further embodiment, the effector is a liposome containing a second pyrimidine-based effector payload. In a further embodiment, the effector is a liposome containing a second nanocarrier-based effector payload. In a further embodiment, the effector is a liposome containing a second complexing agent binding a metallic-based effector payload. In a further embodiment, the effector is a liposome containing a second particulate-based effector payload. In a further embodiment, the effector is a liposome containing a second amino acid-based effector payload. In a further embodiment, the effector is a liposome containing a second non-naturally occurring amino acid-based effector payload. In a further embodiment, the effector is a liposome containing a second oligosaccharide-based effector payload. In a further embodiment, the effector is a liposome containing a second polysaccharide-based effector payload. In a further embodiment, the effector is a liposome containing a second disaccharide-based effector payload. In a further embodiment, the effector is a liposome containing a second amino sugar-based effector payload. In a further embodiment, the effector is a liposome containing a second lipid-based effector payload. In a further embodiment, the effector is a liposome containing a second phospholipid-based effector payload. In a further embodiment, the effector is a liposome containing a second glycolipid-based effector payload. In a further embodiment, the effector is a liposome containing a second sterol-based effector payload. In a further embodiment, the effector is a liposome containing a second hormone-based effector payload. In a further embodiment, the effector is a liposome containing a second neurotransmitter-based effector payload. In a further embodiment, the effector is a liposome containing a second carbohydrate-based effector payload. In a further embodiment, the effector is a liposome containing a second sugar-based effector payload. In a further embodiment, the effector is a liposome containing a second cell-based effector payload. In a further embodiment, the effector is a liposome containing a second killer cell-based effector payload.

In a further embodiment, the effector is a liposome containing a second effector precursor payload to any of the above compounds. In a further embodiment, the effector is a liposome containing a second effector derivative payload to any of the above compounds

In an embodiment, the effector is an ionized fatty acid vesicle. In an embodiment, the effector is micelle. In an embodiment, the effector is a polymeric micelle. In a further embodiment, the effector is a micelle containing a second drug-based effector payload. In a further embodiment, the effector is a polymeric micelle containing a second drug-based effector payload. In a further embodiment, the effector is a micelle containing a second drug-based effector payload wherein the second effector payload is noted in the list above. In a further embodiment, the effector is a polymeric micelle containing a second drug-based effector payload wherein the second effector payload is noted in the list above.

In an embodiment, the effector is a lipoprotein-based drug carrier. In a further embodiment, the effector is a lipoprotein-based drug carrier containing a second drug-based effector payload. In a further embodiment, the effector is a lipoprotein-based drug carrier containing a second drug-based effector payload wherein the second effector payload is noted in the list above.

In an embodiment, the effector is a nano-particle drug carrier. In a further embodiment, the effector is a nano-particle drug carrier containing a second drug-based effector payload. In a further embodiment, the effector is a nano-particle drug carrier containing a second drug-based effector payload wherein the second effector payload is noted in the list above.

In an embodiment, the effector is a dendrimer. In an embodiment, the effector is a dendrimer drug carrier. In a further embodiment, the effector is a dendrimer drug carrier containing a second drug-based effector payload. In a further embodiment, the effector is a dendrimer drug carrier containing a second drug-based effector payload wherein the second effector payload is noted in the list above.

Plasma lipoproteins are transporters of lipids and other hydrophobic molecules in the mammalian circulation. Lipoproteins are spherical yet heterogeneous in particle size, composed of varying percentages of lipid and apolipoprotein. Lipoproteins can be further classified as chylomicrons (CM), very low density lipoprotein (VLDL), low density lipoprotein (LDL), and high density lipoprotein (HDL).

Lipoproteins have a strong potential to serve as drug-delivery vehicles due to their small size, long residence time in the circulation and high-drug payload. Consequently, lipoproteins and synthetic/reconstituted lipoprotein preparations have been evaluated with increasing interest towards clinical applications, particularly for cancer diagnostics/imaging and chemotherapy. For an expert review see (N. Sabnis et al, Ther. Deliv., 2012, 3, 5, 599 and ‘Lipoproteins as Carriers of Pharmacological Agents’, Ed. M. Shaw, CRC Press).

There are several advantages of lipoproteins as anti-tumoural drug carriers: (i) lipoproteins are spherical particles consisting of a core of apolar lipids surrounded by a phospholipid monolayer, in which cholesterol and apoproteins are embedded. Highly lipophilic drugs can be incorporated into the apolar core without affecting lipoprotein receptor recognition; (ii) lipoproteins can be recognized and taken up via specific receptors, and can mediate cellular uptake of the carried drugs; (iii) being endogenous, lipoproteins are completely bio-degradable, do not trigger immunological responses, escape from recognition and elimination by the reticuloendothelial system (RES) and have a relatively long half-life in the circulation; (iv) many cancer cells including the most aggressive ones show a high ability of lipoprotein uptake. This is presumably due to enhanced requirements for structural cholesterol and steroid-derived products in highly proliferative cells.

WO2013125769 describes a composition of a lipoprotein nanocarrier, containing within it a plurality of drug; conjugated to an antibody targeting vehicle for the purpose of development of effective antibody-drug therapeutic agents.

In an embodiment, the effector is a lipoprotein. In an embodiment, the effector is a lipoprotein drug carrier. In a further embodiment, the effector is a lipoprotein drug carrier containing a second drug-based effector payload. In a further embodiment, the effector is a lipoprotein drug carrier containing a second drug-based effector payload wherein the second effector payload is noted in the list above.

In an embodiment, the effector is a chylomicron. In an embodiment, the effector is a chylomicron drug carrier. In a further embodiment, the effector is a chylomicron drug carrier containing a second drug-based effector payload. In a further embodiment, the effector is a chylomicron drug carrier containing a second drug-based effector payload wherein the second effector payload is noted in the list above.

In an embodiment, the effector is a very low density lipoprotein (LPO). In an embodiment, the effector is a very low density lipoprotein (LPO) drug carrier. In a further embodiment, the effector is a very low density lipoprotein (LPO) drug carrier containing a second drug-based effector payload. In a further embodiment, the effector is a very low density lipoprotein (LPO) drug carrier containing a second drug-based effector payload wherein the second effector payload is noted in the list above.

In an embodiment, the effector is a low density lipoprotein (LDL). In an embodiment, the effector is a low density lipoprotein (LDL) drug carrier. In a further embodiment, the effector is a low density lipoprotein (LDL) drug carrier containing a second drug-based effector payload. In a further embodiment, the effector is a low density lipoprotein (LDL) drug carrier containing a second drug-based effector payload wherein the second effector payload is noted in the list above.

In an embodiment, the effector is a high density lipoprotein (HDL). In an embodiment, the effector is a high density lipoprotein (HDL) drug carrier. In a further embodiment, the effector is a high density lipoprotein (HDL) drug carrier containing a second drug-based effector payload. In a further embodiment, the effector is a high density lipoprotein (HDL) drug carrier containing a second drug-based effector payload wherein the second effector payload is noted in the list above.

Antibody-derived agents that engage select subsets of effector cells of the human defence system for the elimination of cancer cells offers great promise as targeted therapy. The recruitment of the body's own cells as effectors for the elimination of malignant cancer cells is exemplified within antibody dependent cell-mediated cytotoxicity (ADCC), antibody dependent cellular phagocytosis (ADCP) and complement-dependent cytotoxicity (CDC) mechanisms. Recruitment of the body's own cells as effectors offers several unique advantages, including a reduced immunogenicity and a greater ease of manufacture of the antibody agents. Most importantly is the recent observation that some cancer cells resist treatment with ADCs; typically cancer stem cells (CSCs) with an intrinsic resistance to the drug or cells with an increased resistance acquired by mutation. Such ADC resistant cells may still be susceptible to agents that recruit cytotoxic effector cells because the intracellular mechanisms by which both classes of agents induce death in cancer cells differ and the mechanisms rendering cells resistant to both classes of agents are not the same. For an expert review see (C. Stein et al, Antibodies, 2012, 1, 1, 88-123).

In an embodiment, the effector is an agent capable of eliciting an ADCP, ADCC or CDC response. In an embodiment, the effector is a moiety capable of presenting a Fc receptor. In an embodiment, the effector is a protein capable of recognition by the Fc domain of a NK cell. In an embodiment, the effector is a protein capable of binding by the Fc domain of a NK cell. In an embodiment, the effector is a T-cell. In an embodiment, the effector is a granulocyte. In an embodiment, the effector is a cytotoxic lymphocyte. In an embodiment, the effector is a NK cell. In an embodiment, the effector is a monocyte. In an embodiment, the effector is a macrophage.

In an embodiment the effector is a cytolytic immunomodulatory protein.

In an embodiment the effector is a protein toxin. In an embodiment the effector is diphtheria toxin. In an embodiment the effector is a pseudomonas endotoxin.

Tumour cells can be efficiently and specific targeted for cell kill in both in-vitro and in-vivo systems using Antibody-Radionucleotide-Conjugates (ARCs). These conjugates couple the affinity and specificity for a cell surface antigen by antibody with a radionuclide payload capable of emitting electrons of varying energies. Furthermore, the same specificity and payload concept can be employed to create an imaging agent or diagnostic tool.

The selection of the optimal radionuclide for radioimmunotherapy (RIT) of cancer depends on the details of the application. For example, if solid tumors are targeted, and if it is assumed that only a minority of cells within the tumour will bind the antibody in sufficiently large amounts then it is necessary to use radionuclide payloads characterised as high-energy particle emitters. Only this type of radiation can kill cells within regions of the tumour that do not take up Abs. However, if every tumour cell can be targeted then other types of radiation may be more appropriate and effective payloads.

Typically, to target a tumour mass, the choice of radionuclide focuses on high energy beta-particles which are intended to kill macroscopic tumour masses. However, such conjugates do not kill single cells or micrometastases efficiently. For killing single cells the use of radionuclides with shorter radiation path lengths is recommended such as those emitting alpha-particles or Auger or conversion electrons.

Typically, effective cell killing with ARCs can be demonstrated only when the targeted antibody reacts with a high-density of antigens upon the malignant cell surface (approximately >106 molecules per cell). Thus, this therapeutic approach is currently limited to very high-density antigens. Nevertheless, the system could be effective with lower density antigens if the approach were optimized. One obvious variable for modification is the radionuclide used. The choice of radionuclide payload will affect both the potency of specific toxicity and the level of non-specific toxicity.

There are currently two marketed anti body-radionuclide-conjugates; ibritumomab tiuxetan (Zevalin®) and tositumomab (Bexxar®), for the treatment of lymphoma, in which radionuclides are targeted to tumours by anti-CD20 antibody. Both of these molecules are generated through conventional conjugation chemistries and techniques.

RACs can also be used in methods such as Immuno-positron emission tomography (ImmunoPET or iPET) to track and quantify antibodies in vivo or for diagnostic purposes (J. Tinianow et al, Nucl. Med. Biol., 2010; 37:289).

Targeted alpha therapy (TAT) is an investigational procedure which utilises monoclonal antibodies (mAbs), peptide conjugates and/or other chemical compounds. These bio-vectors are able to transport a dose of alpha particles to destroy cancer cells. Radionuclide antibody-conjugates (RACs), labelled with beta emitters, have already been used in humans. More recently, TAT has been introduced to treat oncological diseases mainly leukaemia and lymphoma (S. Kitson et al, Curr. Radiopharm., 2013, 6, 2, 57).

In an embodiment, the effector is a radionuclide. In an embodiment, the effector is an alpha particle emitting radionuclide. In an embodiment, the effector is a beta particle emitting radionuclide. In an embodiment, the effector is Auger electron emitting radionuclide. In an embodiment, the effector is a mixed emitting radionuclide.

In an embodiment, the effector is bismuth-213. In an embodiment, the effector is bismuth-212. In an embodiment, the effector is astatine-211. In an embodiment, the effector radium-223.

In an embodiment, the effector is yttrium-90. In an embodiment, the effector is iodine-131. In an embodiment, the effector is samarium-153-EDTMP. In an embodiment, the effector is strontium-89-chloride. In an embodiment, the effector is lutetium-177. In an embodiment, the effector is holmium-166. In an embodiment, the effector is rhenium-186. In an embodiment, the effector is rhenium-188. In an embodiment, the effector is copper-67. In an embodiment, the effector is promethium-149. In an embodiment, the effector is gold-199. In an embodiment, the effector is rhodium-105. Indium-111 Iodine-125 Gallium-67 Samarium-153 Osmium-191 Platinum-193m Platinum-195m Mercury-195m technetium-99m

In an embodiment, the effector is bromine-77. In an embodiment, the effector is indium-111. In an embodiment, the effector is iodine-123. In an embodiment, the effector is iodine-125.

In an embodiment, the effector is a radionuclide complexed with a chelator. In an embodiment, the effector is a radionuclide complexed with a chelating peptide. In an embodiment, the effector is a radionuclide complexed with a somatostatin analog. In an embodiment, the effector is a radionuclide complexed with a chelator peptide-chelator complex. In an embodiment, the effector is a radionuclide complexed with somatostatin analog, octreotide, labeled with 111ln via the chelator diethylenetriaminepentaacetic acid (DTPA). In an embodiment, the effector is Octreoscan.

Typically radionuclide effector moieties are alpha or beta radiation emitters, capable of killing tumour cells or for use as a targeted therapy agent for radioisotope therapy.

Boron Neutron Capture Therapy (BNCT) is a binary radiation therapy that has been used successfully to treat tumours and cancerous cells by non-invasive means. BNCT requires the site specific delivery of a stable, non-radioactive boron-10 isotope to the tumour so that it is concentrated locally to malignant cells. Delivery is achieved by attaching the boron isotope to a ligand or capture agent which in turn is conjugated to a tumour seeking moiety, typically an antibody. Once localised at the tumour the boron-capture agent is then radiated with a beam of epithermal neutrons that interact with the capture agent to produce a biologically destructive nuclear caption reaction. This results in the formation of boron-11 with the release of lethal radiation in the form of alpha particles (helium-4) and lithium ions that kill the tumour. Cells in close proximity to the high energy particulates, primarily cancer cells are killed, leaving adjacent normal cells largely unaffected. Although numerous clinical studies have demonstrated the safety of BNCT, the challenge has been finding more tumour-selective boron delivery agents.

In an embodiment, the effector is boron-10 isotope. In an embodiment, the effector is a boron-10 isotope ligand complex or a boron-10 isotope capture agent complex. In an embodiment, the effector is a biomolecule-boron-10 isotope ligand complex-conjugate for BNCT. In an embodiment, the effector is a biomolecule-boron-10 isotope capture agent complex-conjugate for BNCT. In a further embodiment, the effector is an antibody-boron-10 isotope ligand complex-conjugate for BNCT. In a further embodiment, the effector is an antibody-boron-10 isotope capture agent complex-conjugate for BNCT.

Gadolinium-157 is one of the nuclides that holds interesting properties of being a neutron capture therapy agent. The rationale for the design of Gadolinium Neutron Capture Therapy (GdNCT) agents is to fully exploit the extremely fortuitous combination of ultrahigh neutron capture cross section of Gadolinium-157 and the proton relaxing effect of gadolinium. An effective GdNCT composition would ideally combine diagnostic and therapeutic properties. This would permit the simultaneous dynamic MRI monitoring of the gadolinium distribution levels prior to and during neutron irradiation at therapeutic doses. Identically to BNCT the metal isotope must be delivered selectively to the site of the tumour, typically in the form of a ligand complex or capture agent complex.

In an embodiment, the effector is Gadolinium-157 isotope. In an embodiment, the effector is a Gadolinium-157 isotope ligand complex or a Gadolinium-157 isotope capture agent complex. In an embodiment, the effector is a biomolecule-Gadolinium-157 isotope ligand complex-conjugate for GdNCT. In an embodiment, the effector is a biomolecule-Gadolinium-157 isotope capture agent complex-conjugate for GdNCT. In a further embodiment, the effector is an antibody-Gadolinium-157 isotope ligand complex-conjugate for GdNCT. In a further embodiment, the effector is an antibody-Gadolinium-157 isotope capture agent complex-conjugate for GdNCT.

Nanocarriers are nanometer sized materials (diameter 1-100 nm) that have a high capacity to carry and deliver multiple effector payloads and/or imaging agents. Owing to their high surface-area-to-volume ratio, it is possible to achieve high ligand density on the surface for targeting purposes. Nanocarriers can also be used to increase local effector payload (e.g. drug) concentrations by carrying the effector payload within it and controlling the release when the target is reached. Currently, natural/synthetic polymers and lipids are typically used as drug delivery vectors. Several therapeutic nanocarriers have been approved for clinical use. However, to date, there are only a few clinically approved nanocarriers that incorporate molecules that selectively bind and target tumour cells.

Nanocarriers can offer many advantages over free effector payloads. For example nanocarriers may protect the effector payload from premature degradation and from prematurely interacting with the biological environment. Nanocarriers have been shown to enhance absorption of their payload into a selected tissues such as delivery of a cytotoxic drug to target cell kill of tumour cells as part of tumour mass. Nanocarriers are also proven to control the pharmacokinetic and drug tissue distribution profile and in some circumstances improve intracellular penetration.

The family of nanocarriers includes polymer conjugates, polymeric nanoparticles, lipid-based carriers such as liposomes and micelles, dendrimers, carbon nanotubes, and gold nanoparticles, including nanoshells and nanocages. Nanocarriers have been explored for a variety of applications such as drug delivery, imaging, photothermal ablation of tumours, radiation sensitizers, detection of apoptosis, and sentinel lymphnode mapping. Antibodies, peptides, small molecules and aptamers can be covalently bound to the surface of a nanocarrier enabling a targeting vector for the nanocarrier. An antibody-nanocarrier conjugate can bind to the targeted cell surface through specific antigen recognition by the antibody and upon binding the conjugate can be internalised by receptor-mediated endocytosis (for a comprehensive review see D. Peer et al, Nature Nanotechnology, December 2007, Vol. 2, 751-760).

In an embodiment, the effector is a nanocarrier. In an embodiment the nanocarrier is a polymer. In an embodiment the nanocarrier is a lipid. In an embodiment the nanocarrier is a liposome. In an embodiment the nanocarrier is a micelle. In an embodiment the nanocarrier is a dendrimer. In an embodiment the nanocarrier is are carbon nanotubes. In an embodiment the nanocarrier are gold nanoparticles. In an embodiment the nanocarrier is a nanoshell. In an embodiment the nanocarrier is a nanocage.

In an embodiment the nanocarrier are metal-based particles. In an embodiment the nanocarrier are silica coated micelles (FloDots). In an embodiment the nanocarrier are ceramic formulations. In an embodiment the nanocarrier are perfluorocarbon emulsions. In an embodiment the nanocarrier are magnetic surface-coated nanoparticles. In an embodiment the nanocarrier are semiconductor nanoparticles (quantum dots). In an embodiment the nanocarrier are phospholipids. In an embodiment the nanocarrier is chitosan. In an embodiment the nanocarrier is dextran. In an embodiment the nanocarrier is lactic acid. In an embodiment the nanocarrier is cross-linked liposomes.

Biomolecule-Effector-Conjugates:

In accordance with the present invention there is provided a biomolecule-effector-conjugate obtainable by a process of the present invention.

Biomolecule-Reporter-Conjugates:

In accordance with the present invention there is provided a biomolecule-reporter-conjugate obtainable by a process of the present invention.

The examples of PCT publication WO2014/174316 relate to the preparation of antibody drug conjugates. The drug portion of the ADCs of these examples can be substituted for an effector or reporter group as hereinbefore described. Thus, the biomolecule-effector-conjugates and biomolecule-reporter-conjugates of the present invention can be manufactured by analogous methods described in WO2014/174316.

1. A method of synthesizing a biomolecule-effector-conjugate, the method comprising:
a) optionally contacting a biomolecule with an agent selected from the group consisting of a chemical modification agent, an enzymatic modification agent, and an activating agent to provide a chemically modified, enzymatically modified, or activated biomolecule;
b)
(i) when step (a) is carried out, contacting the chemically modified, enzymatically modified, or activated biomolecule of step (a) with a capture resin comprising a non-peptide based capture moiety selected from the group consisting of Protein A, Protein G, and Protein L mimetic biomolecule capture moiety under conditions suitable to immobilise the chemically modified, enzymatically modified, or activated biomolecule and therefore provide an immobilised chemically modified, enzymatically modified, or activated biomolecule; or
(ii) when step (a) is not carried out, contacting a biomolecule with a capture resin comprising a non-peptide based capture moiety selected from the group consisting of Protein A, Protein G, and Protein L mimetic biomolecule capture moiety under conditions suitable to immobilise the biomolecule and therefore provide an immobilised biomolecule;
c) optionally contacting the immobilised chemically modified, enzymatically modified, or activated biomolecule of step (b)(i); or the immobilised biomolecule of step (b)(ii) with an agent selected from the group consisting of a chemical modification agent, an enzymatic modification agent, and an activating agent to provide an immobilised chemically modified, enzymatically modified, or activated biomolecule;
d) optionally washing the immobilised chemically modified, enzymatically modified, or activated biomolecule of step (b)(i); the immobilised biomolecule of step (b)(ii); or the immobilised chemically modified, enzymatically modified or activated, immobilised biomolecule of step (c) with buffer to remove superfluous or unreacted chemical modification agent, enzymatic modification agent, or activating agent,
e) optionally repeating step (c) and step (d);
f) optionally contacting an effector component with an agent selected from the group consisting of a chemical modification agent, an enzymatic modification agent, and an activating agent to provide a chemically modified, enzymatically modified, or activated effector component;
g)
(i) when step (f) is carried out, contacting the immobilised biomolecule or the immobilised chemically modified, enzymatically modified, or activated biomolecule with the chemically modified, enzymatically modified, or activated effector component of step (f) to form an immobilised biomolecule-effector-conjugate; or
(ii) when step (f) is not carried out contacting the immobilised biomolecule or the immobilised chemically modified, enzymatically modified, or activated biomolecule with an effector component to form an immobilised biomolecule-effector-conjugate;
h) optionally washing the immobilised biomolecule-effector-conjugate of step (g) with buffer to remove superfluous or unreacted reagents, to provide a purified immobilised biomolecule-effector-conjugate;
i) releasing the purified biomolecule-effector-conjugate from the capture resin; wherein the biomolecule is selected from the group consisting of an antibody, a modified antibody, and an antibody fragment.

2. A method of synthesizing a biomolecule-reporter-conjugate, the method comprising:
a) optionally contacting a biomolecule with an agent selected from the group consisting of a chemical modification agent, an enzymatic modification agent, and an activating agent to provide a chemically modified, enzymatically modified, or activated biomolecule;
b)
(i) when step (a) is carried out, contacting the chemically modified, enzymatically modified, or activated biomolecule of step (a) with a capture resin comprising a non-peptide based capture moiety selected from the group consisting of Protein A, Protein G, and Protein L mimetic biomolecule capture moiety under conditions suitable to immobilise the chemically modified, enzymatically modified, or activated biomolecule and therefore provide an immobilised chemically modified, enzymatically modified, or activated biomolecule; or
(ii) when step (a) is not carried out, contacting a biomolecule with a capture resin comprising a non-peptide based capture moiety selected from the group consisting of Protein A, Protein G, and Protein L mimetic biomolecule capture moiety under conditions suitable to immobilise the biomolecule and therefore provide an immobilised biomolecule;
c) optionally contacting the immobilised chemically modified, enzymatically modified, or activated biomolecule of step (b)(i) or the immobilised biomolecule of step (b)(ii) with an agent selected from the group consisting of a chemical modification agent, an enzymatic modification agent, and an activating agent to provide an immobilised chemically modified, enzymatically modified, or activated biomolecule;
d) optionally washing the immobilised chemically modified, enzymatically modified, or activated biomolecule of step (b)(i); the immobilised biomolecule of step (b)(ii); or the immobilised chemically modified, enzymatically modified or activated, immobilised biomolecule of step (c) with buffer to remove superfluous or unreacted chemical modification agent, enzymatic modification agent, or activating agent,
e) optionally repeating step (c) and step (d);
f) optionally contacting an effector component with an agent selected from the group consisting of a chemical modification agent, an enzymatic modification agent, and an activating agent to provide a chemically modified, enzymatically modified, or activated effector component;
g)
(i) when step (f) is carried out, contacting the immobilised biomolecule or the immobilised chemically modified, enzymatically modified, or activated biomolecule with the chemically modified, enzymatically modified, or activated reporter component of step (f) to form an immobilised biomolecule-reporter-conjugate; or
(ii) when step (f) is not carried out contacting the immobilised biomolecule or the immobilised chemically modified, enzymatically modified, or activated biomolecule with a reporter component to form an immobilised biomolecule-reporter-conjugate;
h) optionally washing the immobilised biomolecule-reporter-conjugate of step (g) with buffer to remove superfluous or unreacted reagents, to provide a purified immobilised biomolecule-reporter conjugate;
i) releasing the purified biomolecule-reporter-conjugate from the capture resin; wherein the biomolecule is selected from the group consisting of an antibody, a modified antibody, and an antibody fragment.

3. (canceled)

4. The method of claim 1, wherein step (a) is carried out.

5. The method of claim 4, wherein step (a) involves reducing the biomolecule.

6. The method of claim 4, wherein step (a) involves reacting the biomolecule with a crosslinker moiety, optionally wherein the crosslinker moiety is an amine-to-sulfhydryl crosslinker.

7. The method of claim 6, wherein the crosslinker moiety is succinimidyl 4-(N-maleimidomethyl)cyclohexane-1-carboxylate (SMCC).

8. The method of claim 1, wherein step (b) involves incubation with the capture resin, optionally wherein the incubation is carried out at temperature of from about 5° C. to about 50° C. or optionally wherein the incubation is for a period of time of from about 10 minutes to about 18 hours.

9. (canceled)

10. The method of claim 1, wherein step (c) is carried out.

11. The method of claim 10, wherein step (c) involves reducing the biomolecule.

12. The method of claim 10, wherein step (c) involves reacting the biomolecule with a crosslinker moiety, optionally wherein the crosslinker moiety is an amine-to-sulfhydryl crosslinker.

13. The method of claim 12, wherein the crosslinker moiety is succinimidyl 4-(N-maleimidomethyl)cyclohexane-1-carboxylate (SMCC).

14. The method of claim 1, wherein step (d) is omitted.

15. The method of claim 1, wherein step (d) is carried out, optionally wherein the washing involves rinsing with a buffer, further optionally wherein the buffer is phosphate buffered saline (PBS).

16. (canceled)

17. The method of claim 1, wherein step (f) is carried out.

18. The method of claim 17, wherein the step of contacting the reporter or effector component with a chemical modification agent, an enzymatic modification agent, or an activating agent to provide a modified or activated effector component involves reacting the effector component with a crosslinker moiety, optionally wherein the crosslinker moiety is an amine-to-sulfhydryl crosslinker.

19. The method of claim 18, wherein the crosslinker moiety is succinimidyl 4-(N-maleimidomethyl)cyclohexane-1-carboxylate (SMCC).

20. The method of claim 1, wherein step (g) involves simultaneously (1) carrying out the chemical modification, enzymatic modification, or activation of the effector component and (2) contacting the immobilised biomolecule or the chemically modified, enzymatically modified, or activated, immobilised biomolecule.

21-22. (canceled)

23. The method of claim 1, wherein step (h) is carried out, optionally wherein the washing involves rinsing with a buffer, further optionally wherein the buffer is phosphate buffered saline (PBS).

24. The method of claim 1, wherein step (i) involves a) exposing the support-biomolecule compound to a release agent; or b) altering the pH to break the support-biomolecule bond.

25. The method of claim 1, wherein the capture resin is selected from the group consisting of Fabsorbent™ F1P HF resin, Mabsorbent™ A1P resin, and Mabsorbent™ A2P resin.
